(19) United States
(12) Patent Application Publication
(10) Pub. No.: US 2010/0217003 A1
(43) Pub. Date: Aug. 26, 2010

(54) 1,2,3,4-TETRAHYDROISOQUINOLINE DERIVATIVES HAVING EFFECTS OF PREVENTING AND TREATING DEGENERATIVE AND INFLAMMATORY DISEASES

(76) Inventors: On-You Hwang, Seoul (KR); Dae-Yoon Chi, Seoul (KR); Hyo-Jin Son, Seoul (KR); Jai-Woong Seo, Incheon (KR)

Correspondence Address: SHERR & VAUGHN, PLLC, 620 HERNDON PARKWAY, SUITE 320, HERNDON, VA, 20170, US

(21) Appl. No.: 12/518,068

(22) PCT Filed: Dec. 10, 2007
(86) PCT No.: PCT/KR2007/006385

§ 371 Date: Jun. 5, 2009

(30) Foreign Application Priority Data
Dec. 8, 2006 (KR) 10-2006-0124270

(51) Int. Cl. C07D 217/04

(52) U.S. Cl. 546/146; 546/150

(57) ABSTRACT

Provided are 7-hydroxy-6-methoxy-1,2,3,4-tetrahydroisoquinoline derivatives and synthesis methods thereof. The compounds significantly inhibit the production of nitrogen monoxide (NO) and superoxide in an activated microglial cell and expressions of TNF-α, IL-1β inducive NO synthase and cyclooxygenase-2 genes. They also prevent NF-kB shift to a nucleus, decrease reactive oxygen species (ROS), inhibit expression of GTP cyclohydrolase I gene and over-production of tetrahydrobiopterin (BH₄), and protect dopaminergic neurons from injury due to activated microglial cells. Consequently, the compounds are effective in treating inflammatory and neurodegenerative diseases.

### TECHNICAL FIELD

The present invention relates to 7-hydroxy-6-methoxy-1,2,3,4-tetrahydroisoquinoline derivatives having effects of preventing and treating degenerative and inflammatory diseases.

### BACKGROUND ART

Recent studies show an inflammation response to be one of the critical mechanisms causing neurodegenerative diseases. Microglial cells, immune cells present in the central nervous system, may be activated by exogenous or endogenous substances so as to produce and release substances such as inflammatory cytokine, TNF-α or IL-1β carbon monoxide (NO), prostaglandin, superoxide, and so forth. Although they induce an immune reaction in the short term, such substances are continuously produced to excess, thereby leading to the loss of adjacent neurons and finally causing neurode-generative diseases. Moreover, the substances released from dying neurons induce re-activation of the microglial cells, so the neurodegenerative diseases go from bad to worse. It has been reported that the activation of the microglial cells is linked to various neurodegenerative diseases, for example, Alzheimer's disease, Parkinson's disease, Huntington's disease, Lou Gehrig's disease, Creutzfelt-Jakob's disease (CJD), etc. Accordingly, it is expected that inhibition of the production of various inflammatory substances released from the activated microglial cells will be very effective in preventing and/or treating neurodegenerative diseases. This is a hot topic of research worldwide.

Currently, the therapy for Parkinson's disease is focused on relief of movement disorder by administering a dopamine precursor, L-DOPA. Unfortunately, the administration of L-DOPA may not enable Parkinson's disease patients to lead normal lives, and it can also cause a variety of chronic physical and mental side-effects. Further, there is an evidence for the neurotoxicity of L-DOPA itself. There is still little known about the treatment and prevention of degeneration by Parkinson's disease. A pharmaceutical method for treating Alzheimer's disease is now based on an acetylcholine esterase inhibitor or Meantime, or an N-methyl-D-aspartate channel blocker. Although there have been attempts to develop various substances such as a secretase inhibitor, no such developed substances have been clinically tested. Moreover, no effective method for treating other neurodegenerative diseases such as Lou Gehrig's disease, Creutzfeldt-Jakob's disease and Huntington's diseases has been developed yet. Thus, there is an urgent need to develop more effective methods for treating such diseases on the basis of their causes.

### DISCLOSURE OF INVENTION

### Technical Problem

The present invention is directed to a novel compound inducing down-regulation in production of various inflammatory cytokines and toxic substances in activated microglial cells.

The present invention is also directed to a novel compound preventing neuron injury from oxidative stress.

The present invention is also directed to a method for synthesizing a novel compound effective in preventing and/or treating various neurodegenerative and inflammatory diseases.

### Technical Solution

In one aspect, a 7-hydroxy-6-methoxy-1,2,3,4-tetrahydroisoquinoline derivative (Formula 1) for preventing and treating neurodegenerative diseases is provided.

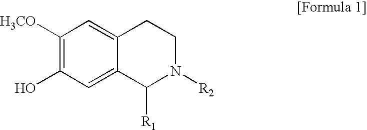

Here, R₁ is selected from the group consisting of H, CH₃, CH₂CH₃, CH₂CH₂CH₃, CH(CH3)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH₂CH₃, CH₂CH₂CH₂CH₃, CH₂Ph, CH₂CH₂Ph, COCH₃(Ac), COCH₂CH₃, COCH₂CH₂CH₃, COCH(CH₃)COCH₂CH(CH₃)₂, cyclohexylmethyl and cyclohexanecarbonyl.

In another aspect, a 7-hydroxy-6-methoxy-1,2,3,4-tetrahydroisoquinoline derivative (Formula 1) for preventing and treating inflammatory diseases is provided.

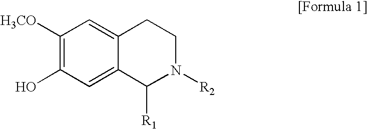

Here, R₁ is selected from the group consisting of H, CH₃, CH₂CH₃, CH₂CH₂CH₃, CH(CH3)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH₂CH₃, CH₂CH₂CH₂CH₃, CH₂Ph, CH₂CH₂Ph, COCH₃(Ac), COCH₂CH₃, COCH₂CH₂CH₃, COCH(CH₃)₂, OCH₂CH(CH₃)₂, cyclohexylmethyl and cyclohexanecarbonyl.

In still another aspect, a 7-hydroxy-6-methoxy-1,2,3,4-tetrahydroisoquinoline derivative (Formula 1) effective in protection of neurons is provided.

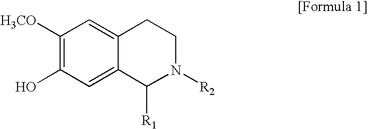

Here, R₁ is selected from the group consisting of H, CH₃, CH₂CH₃, CH₂CH₂CH₃, CH(CH3)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH₂CH₃, CH₂CH₂CH₂CH₃, CH₂Ph, CH₂CH₂Ph, COCH₃(Ac), COCH₂CH₃, COCH₂CH₂CH₃, COCH(CH₃)₂, OCH₂CH(CH₃)₂, cyclohexylmethyl and cyclohexanecarbonyl.

Specific application examples for the ligands, R₁ and R₂ are as follows.

TABLE 1

| Chemical Formula No. | R1 | R2 |
|---|---|---|
| 2 | H | Ac |
| 5a | CH₃ | Ac |
| 9a | CH₂CH₃ | Ac |
| 9a | CH₂CH₂CH₃ | Ac |
| 9c | CH(CH₃)₂ | Ac |
| 9e | CH₂CH(CH₃)₂ | Ac |
| 5b | Ph | Ac |
| 9d | CH₂Ph | Ac |
| 9f | cyclopropyl | Ac |
| 9g | cyclobutyl | Ac |
| 9h | cyclohexyl | Ac |
| 11a | H | COCH₂CH₃ |
| 11b | H | COCH₂CH₂CH₃ |
| 11d | H | COCH(CH₃)₂ |
| 11e | H | COCH₂CH(CH₃)₂ |
| 11c | H | cyclohexanecarbonyl |
| 12d | H | CH₂CH₃ |
| 12a | H | CH₂CH₂CH₃ |
| 12b | H | CH₂CH₂CH₂CH₃ |
| 12c | H | cyclohexylmethyl |
| 12e | H | CH₂Ph |
| 12f | H | CH₂CH₂Ph |

The compounds used herein may be either trans or cis configuration.

The 7-hydroxy-6-methoxy-1,2,3,4-tetrahydroisoquinoline (HMTIQ) derivative (Formula 1) may be synthesized by the method as follows.

Several alkyl derivatives were introduced in C1 position by Pictet-Spengler cy-clization using aldehyde and phenylacetylamine (Equation 1). That is, by cyclization of a compound of Formula 3 with acetaldehyde in acid medium, a 1-methyl compound (Formula 4a) may be easily obtained, but 1-phenyl compound (Formula 4b) may not be because of the solubility of benzaldehyde in an aqueous solvent. Thus, by an alternative method, after cyclization in methanol to produce imine, triplefluoroacetic acid (TFA) was added for cyclization with imine, thereby yielding the compound of Formula 4b.

Compounds of Formulae 5a and 5b were synthesized by acetylation of the compounds of Formulae 4a and 4b with acetic anhydride.

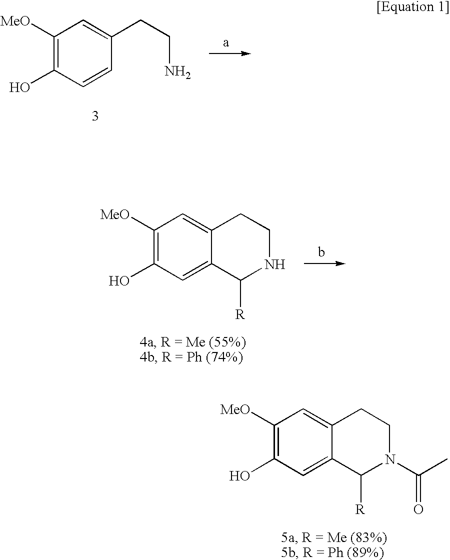

Conditions for synthesis of compound of Formula 4a in Equation 1: acetaldehyde, 1M HCl, 100° C., 24 h;

Conditions for synthesis of compound of Formula 4b in Equation 1: i. benzaldehyde, MgSO₄, TEA, MeOH, reflux, 3 h; ii. TFA, 80° C., 1 h 40 min; and

Conditions for synthesis of compound of Formulae 5a and 5b in Equation 1: (b) Ac₂O, Et₃N, CH₂Cl₂, room temperature (RT), 1 h.

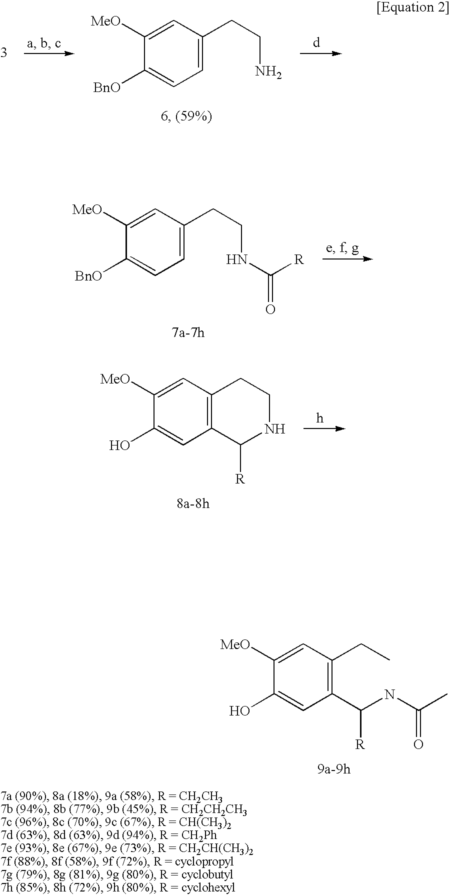

Conditions for synthesis of compound of Formula 6 in Equation 2: (a) (Boc)₂O, Et₃N, CHCl₃, RT, 24 h; (b) benzyl-bromide, K₂CO₃, acetone, reflux, 12 h; (c) TFA, CH₂Cl₂, 0° C., 40 min;

Conditions for synthesis of compound of Formulae 7a-7h in Equation 2: acyl chloride, TEA, CH₂Cl₂, RT, 30 min-1 h;

Conditions for synthesis of compound of Formulae 8a-8h in Equation 2: (e) POCl₃, CH₃CN, reflux, 2-5 h; (f) NaBH₄, 0° C.-RT, 24 h; (g) Pd/C, H₂, HCl, MeOH, RT, 12 h; and

Conditions for synthesis of compound of Formulae 9a-9h in Equation 2: (h) Ac₂O, CH₂Cl₂, 0° C.-RT, 2 h.

Additional synthesis of tetrahydroisoquinoline derivatives in C-1 position was performed by Bischler-Napieralski reaction. A compound of Formula 6 may be synthesized by protecting a primary amine and phenol with tert-butyloxycarbonyl anhydride and benzyl-bromide, respectively, and detaching a tert-butyloxycarbonyl group. Amine derivatives (Formulae 7a-7h) may be synthesized by acylation of the compound of Formula 6 with several acyl chlorides such as propionyl, butyryl, isobutyryl, α-phenylacetyl, 4-methylbutyryl, cyclopropanecarbonyl, cyclobutanecarbonyl and cyclohexanecarbonyl chlorides.

These acylated compounds (Formulae 7a-7h) may be treated with phosphorus oxychloride to obtain cyclic dihydroisoquinoline, which may be reduced with sodium cyanoborohydride to synthesize 7-benzyloxytetrahydroisoquinoline derivatives. Tetrahydroisoquinoline derivatives (Formulae 8a-8h) to which several C-1 alkyl groups were introduced as hydrochloride salt were yielded by palladium-catalyzed debenzylation.

The resultant compounds reacted with acetic anhydrides to produce target compounds of Formulae 9a-9h. All C1-substituted tetrahydroisoquinoline derivatives (Formulae 5a-5b, and 9a-9h) were isolated as racemic mixtures.

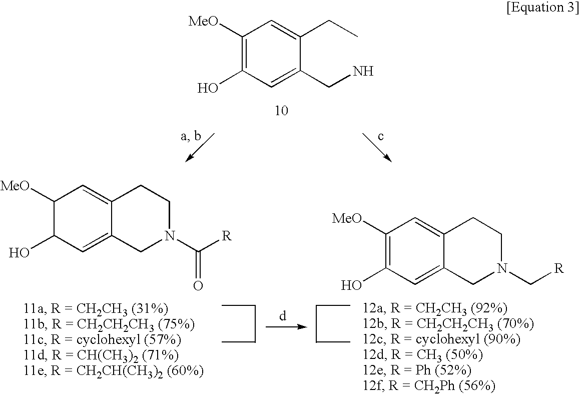

Conditions for synthesis of compounds of Formulae 11a-11e in Equation 3: (a) propionic anhydride, butyryl chloride, cyclohexanecarbonyl chloride, isobutyryl chloride or 3-methylbutyryl chloride, TEA, CH₂Cl₂, RT, 1 h; (b) K₂CO₃, MeOH, reflux, 2 h;

Conditions for synthesis of compounds of Formulae 12d-12f in Equation 3: (c) i. acetaldehyde, benzaldehyde, or phenylacetylaldehyde, Ti(OiPr)₄, EtOH, RT, 1 h, ii. NaCNBH₃, THF, RT, 20 h;

Conditions for synthesis of compounds of Formulae 12a-12c in Equation 3: (d) LA H, THF, reflux, 3-5 h.

N2-acyl derivatives (Formulae 11a-11e) and their carbonyl-reduced derivatives (Formulae 12a-12f) were synthesized.

7-hydroxy-6-methoxy-1,2,3,4-tetrahydroisoquinoline (Formula 10) was prepared by a well-known method in the art [Seo, J. W.; Srisook, E.; Son, H. J.; Hwang, O.; Cha, Y. N.; Chi, D. Y., Bioorg. Med. Chem. Lett. 2005, 15, 3369]. Several N2-carbonylalkyltetrahydroisoquinoline (Formulae 11a-11e) were synthesized by reacting acyl chloride (butyryl chloride, cyclohexanecarbonyl chloride, isobutyryl chloride or 3-methylbutylyl chloride) or its anhydride (propionic anhydride) with triethylamine in a dichloromethane solvent at RT, extracting the mixture, and refluxing the mixture with potassium carbonate in a methanol solvent.

N2-alkyl derivatives (Formulae 12a-12f) were synthesized by two different methods. N2-ethyl, propyl and cyclohexyl tetrahydroisoquinolines (Formulae 12a-12c) were formed by reducing the amides (Formulae 11a-11c) with lithium aluminum hydride, and other tertiary amine derivatives (Formulae 12d-12f) may be synthesized by reacting acetaldehyde, benzaldehyde or phenylacetylaldehyde with titanium(IV) isopropoxide to form imine, and adding sodium cyanoborohydride.

The HMTIQ derivatives described above have the effects of down-regulating various inflammatory cytokines and inflammation-inducing substances in activated microglial cells, protecting neurons from oxidative and inflammatory injuries, and preventing and/or treating neurodegenerative diseases.

Accordingly, the HMTIQ derivatives or their pharmaceutically available salts are used to prevent and treat neurodegenerative and inflammatory diseases.

Further, the present invention may provide a pharmaceutical composition for preventing and treating neurodegenerative and inflammatory diseases, which includes the HMTIQ derivative or its pharmaceutically available salt and a pharmaceutically available diluent or carrier.

### ADVANTAGEOUS EFFECTS

According to the present invention, 7-hydroxy-6-methoxy-1,2,3,4-tetrahydroisoquinoline derivatives significantly inhibit increases of nitrogen monoxide (NO) and superoxide in activated microbial cells, expression of TNF-α, IL-1β inductive NO synthase and cyclooxyganase-2 genes, and the shift of NF-kB to a nucleus, and reduce production of ROS, inhibit expression of a GTP cyclohydrolase I gene and overproduction of tetrahydrobiopterin (BH₄), and significantly protect dopaminergic neurons from damage caused by activated microglial cells.

### BRIEF DESCRIPTION OF THE DRAWINGS

FIG. 1 is a graph illustrating the inhibitory effect of N-ethylcarbonyl-7-hydroxy-6-methoxy-1,2,3,4-tetrahydroisoquinoline (EHMTIQ) on an NO productionproduction in an activated microglial cell.

FIG. 2 is a graph illustrating the inhibitory effect of EHMTIQ on a superoxide productionproduction in an activated microglial cell.

FIG. 3 illustrates the inhibitory effect of EHMTIQ on a quantitative increase of TNF-α mRNA in an activated microglial cell: A) is a photograph of agarose gel electrophoresis of RT-PCT products; and B) is a graph of TNF-α band intensities measured by a densitometer.

FIG. 4 illustrates the inhibitory effect of EHMTIQ on a quantitative increase of IL-1β mRNA in an activated microglial cell: A) is a photograph of agarose gel electrophoresis of RT-PCR products, and B) is a graph of IL-1β band intensities measured by a densitometer.

FIG. 5 illustrates the inhibitory effect of EHMTIQ on a quantitative increase of COX-2 mRNA in an activated microglial cell: A) is a photograph of agarose gel electrophoresis of RT-PCR products; and B) is a graph of COX-2 band intensities measured by a densitometer.

FIG. 6 illustrates the inhibitory effect of EHMTIQ on a quantitative increase of iNOS mRNA in an activated microglial cell: A) is a photograph of agarose gel electrophoresis of RT-PCR products; and B) is a graph of iNOS band intensities measured by a densitometer.

FIG. 7 illustrates the inhibitory effect of EHMTIQ on a quantitative increase of GTPCH mRNA in an activated microglial cell: A) is a photograph of agarose gel electrophoresis of RT-PCR products; and B) is a graph of GTPCH band intensities measured by a densitometer.

FIG. 8 is a graph illustrating the inhibitory effect of EHMTIQ on NF-kB p65 shift to the nucleus in an activated microglial cell.

FIG. 9 is a graph illustrating the inhibitory effect of EHMTIQ on accumulation of oxidative substances in an activated microglial cell.

FIG. 10 is a graph illustrating the inhibitory effect of EHMTIQ on dopaminergic neuron injury by substances released from an activated microglial cell.

FIG. 11 is a graph illustrating the stability of EHMTIQ to degradation induced by microsomal enzymes.

FIG. 12 is a graph illustrating the inhibitory effect of 7-hydroxy-6-methoxy-1,2,3,4-tetrahydroisoquinoline (HMTIQ) on a BH₄ production in an activated microglial cell.

FIG. 13 illustrates microglial cells immunostained for the microglial marker, Iba-1, which show the inhibitory effect of EHMTIQ on the activation of microglial cells in the substantia nigra of a mouse model of Parkinson's disease induced by MPTP.

FIG. 14 illustrates dopaminergic neurons immunostained for the dopaminergic neuronal marker, tyrosine hydroxylase (TH), which show the protective effect of EHMTIQ on dopaminergic neurons in the substantia nigra of a mouse model of Parkinson's disease induced by MPTP (scale bar=200 [symbol] ).

Here, two panels on the right are photographs of double-labeled dopaminergic neurons with fluoroJade C and TH, which show the inhibitory effect of EHMTIQ on de-generation of dopaminergic neurons in the substantia nigra (scale bar=20 [symbol] )

FIG. 15 illustrates dopaminerginc neurons immunostained for TH, which show the protective effect of EHMTIQ on dopamine nerve terminals in the striatum of a mouse model of Parkinson's disease induced by MTPT (scale bar=50 [symbol] ).

FIG. 16 illustrates non-toxicity of EHMTIQ to a mouse liver cell (scale bar=100 [symbol] ).

### BEST MODE FOR CARRYING OUT THE INVENTION

Hereinafter, methods of synthesizing intermediates and structural analysis of final products (5a, 5b, 9a-9h, 11a-, 11e and 12a-12f) having the aforementioned effects will be described.

### Exemplary Embodiment 1

### Synthesis of 2-acetyl-7-hydroxy-6-methoxy-1,2,3,4-tetrahydroisoquinoline (AHMTIO) Derivatives, Substituted with Methyl or Phenyl in C1 Position and Analysis of their Structures

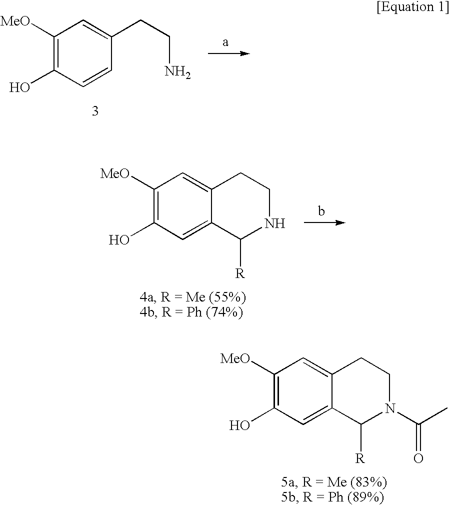

Preparation and Analysis of AHMTIQ (5a)

Acetaldehyde (15.7 mmol, 692 mg) was reacted with 3-O-methyl dopamine hydrochloride (1.96 mmol, 400 mg) dissolved in 1M HCl solution (10 ml) in a pressure tube for 24 hours at 100° C. The reaction tube was cooled and the mixture was neutralized with sodium bicarbonate. Water and the remaining solvent were removed under reduced pressure and the result was dried in a vacuum. Methanol was added to filter remaining precipitate and crude compound 4a was extracted by short column chromatography. The crystallization of the crude compound 4a yielded a white powdery hydrochloride salt (250 mg, 55%) [¹H NMR (DMSO-d₆, 400 MHz) δ9.93 (br s, 1H), 9.40 (br s, 1H), 9.06 (s, 1H), 6.72 (s, 1H), 6.67 (s, 1H), 4.32-4.30 (m, 1H), 3.47 (s, 3H), 3.28-3.36 (m, 1H), 3.21-3.22 (m, 1H), 2.29-2.00 (m, 1H) 1.52 (d, J=6.8 Hz, 3H); ¹³C NMR (DMSO-d₆, 100 MHz) δ147.1, 145.4, 126.0, 122.1, 112.7, 111.9, 55.6, 49.7, 38.6, 24.6, 19.1; MS (CI) 194 (M⁺+1, 100), 178, 164].

The intermediate 4a (0.435 mmol, 100 mg) was added to a dichloromethane solvent (5 ml), and acetic anhydride (0.435 mmol, 45 mg) and triethylamine (1.0 mmol) were added thereto at room temperature (RT). After stirring the resultant mixture for one hour, remaining solvent was removed under reduced pressure. A saturated sodium bi-carbonate solution was poured and the mixture was extracted with a dichloromethane solvent. Recrystallization yielded white compound 5a (91 mg, 83%) [¹H NMR (CDCl₃, 400 MHz) δ6.96 (s, one conformer of C5-H), 6.66 (s, one conformer of C5-H), 6.59 (s, one conformer of C8-H), 6.57 (s, one conformer of C8-H), 5.83 (s, one conformer of O—H), 5.78 (s, one conformer of O—H), 5.53 (q, J=6.6 Hz, one conformer of C1-H), 4.83 (q, J=6.6 Hz, one conformer of C1-H), 4.70-4.65 (m, one conformer of C3-H), 3.82-3.76 (m, one conformer of C3-H), 3.52-3.44 (m, one conformer of C3-H), 3.02-2.95 (m, one conformer of C3-H), 2.90-2.79 (m, 1H), 2.75-2.70 (m, one conformer of C4-H), 2.69-2.60 (m, one conformer of C4-H), 2.18 (s, one conformer of C1-CH₃), 2.15 (s, one conformer of C1-CH₃), 1.49 (d, J=6.4 Hz, one conformer of COC—H₃), 1.40 (d, J=6.8 Hz, one conformer of COC—H₃); ¹³C NMR (CDCl₃, 100 MHz) δ168.8, 168.7, 145.5, 145.3, 144.3, 144.1, 131.3, 130.0, 125.6, 124.4, 112.8, 112.3, 110.8, 110.4, 55.9, 52.3, 47.9, 40.4, 34.8, 29.0, 28.2, 22.5, 21.9, 21.5, 21.4; MS (EI): 471, 264, 236 (M⁺+100), 220].

Preparation and Analysis of AHMTIO (5b)

Compound 3 (1.0 mmol, 204 mg), magnesium sulfate (2.49 mmol, 300 mg), benzaldehyde (1.0 mmol, 106 mg) and triethylamine (2.0 mmol, 202 mg) were added to methanol absolute (15 ml) and refluxed for 3 hours. After the reaction container was cooled, the mixture was filtered with cellite. The solvent was removed under reduced pressure and the mixture was filtered with ethylacetate to remove a white precipitate. The filtered solution was re-filtered and vacuum-dried under reduced pressure. Trifluoroacetic acid was added to the dried result, refluxed for one hour, and removed of the solvent under reduced pressure. The result was neutralized with sodium bicarbonate and then extracted with dichloromethane.

The organic layer was dried with sodium sulfate and the solvent was removed under reduced pressure. Intermediate 4b (188 mg, 74%) was yielded by column chromatography (5% methanol, 95% dichloromethane) [¹H NMR (DMSO-d₆, 400 MHz) δ8.57 (br s, 1H), 7.22-7.32 (m, 5H), 6.63 (s, 1H), 6.04 (s, 1H), 3.72 (s, 3H), 3.01-3.10 (m, 1H), 2.76-2.89 (m, 2H), 2.55-2.65 (m, 1H); ¹³C NMR (DMSO-d₆, 100 MHz) 6146.0, 145.6, 144.1, 130.7, 128.9, 128.0, 126.8, 125.7, 114.4, 112.2, 60.8, 55.5, 42.0, 28.8; MS (CI): 284, 256 (M⁺+1, 100), 178].

The intermediate 4b (0.223 mmol, 57 mg) was added to chloroform (5 ml), and acetic anhydride (0.223 mmol, 23 mg) was further added thereto at RT. After stirring the mixture for one hour, the solvent was removed under reduced pressure and saturated sodium bicarbonate was added. Following extraction with dichloromethane and removal of the solvent, white solid 5b (59 mg, 89%) was yielded by recrystallization [¹H NMR (CDCl₃, 400 MHz) δ7.32-7.18 (m, 5H), 6.86-6.58 (m, several conformer peak of C5-H and C8-H, H3), 5.84-5.30 (m, several conformer peak of O—H, 1H), 4.33-4.27 (m, one conformer of C1-H), 3.89 (s, 3H) 3.72-3.66 (m, one conformer of C1-H), 3.46-3.38 (m, one conformer of C3-H), 3.27-3.21 (m, one conformer of C3-H), 2.96-2.82 (m, one conformer of C4-H) 2.76-2.60 (m, one conformer of C4-H) 2.74 (s, one conformer of COC—H₃), 2.15 (s, one conformer of COC—H); ¹³C NMR (CDCl₃, 100 MHz) δ169.7, 168.9, 145.8, 144.2, 143.9, 142.4, 141.4, 128.6, 129.5, 128.1, 127.8, 127.7, 127.5, 127.2, 126.8, 125.7, 114.5, 113.8, 110.8, 110.4, 60.2, 55.9, 54.4, 40.4, 37.6, 28.6, 27.4, 22.1, 21.7 MS (EI); 297 (M⁺), 254, 239, 220, 178 (100), 163].

### Exemplary Embodiment 2

### Synthesis Method of AHMTIQ Derivatives (6, 7a-h, 8a-h and 9a-g) Substituted with Several Alkyls in C1 Position and Analysis of their Structures

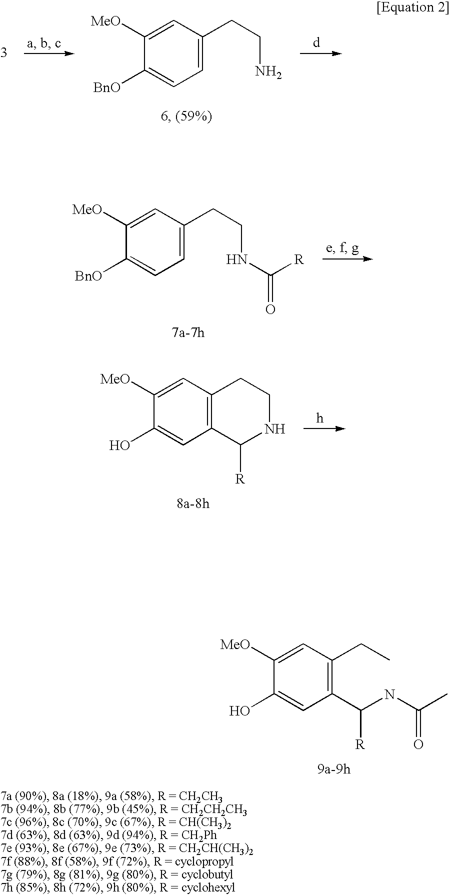

\textcircled{1} Synthesis Method and Analysis of N-(2-(4-hydroxy-3-O-methylphenyl)ethyl)-tertiary butyl carbonate (6)

Method a) Tertiary butyl oxycarbonyl anhydride (7.63 mmol, 1.67 g) and triethylamine (19.5 mmol, 1.93 g) were added to chloroform (20 ml) with compound 3 (6.35 mmol, 1.30 g). The mixture was stirred for 24 hours at RT and aluminum chloride solution was added thereto. The mixture was extracted with a dichloromethane solvent and then the organic layer was washed twice with water. A white crystalline compound (1.32 g, 59%) was yielded by column chromatography and recrystallization [¹H NMR (CDCl₃, 200 MHz) δ6.83 (d, J=8.4 Hz, 1H), 6.63-6.67 (m, 2H), 5.83 (s, 1H), 6.45 (br s, 1H), 3.84 (s, 3H), 3.35 (q, J=6.6 Hz, 2H), 2.70 (t, J=7.0 Hz, 2H), 1.43 (s, 9H); ¹³C NMR (CDCl₃, 50 MHz) δ155.9, 146.5, 144.1, 130.6, 121.2, 114.4, 111.2, 79.1, 55.7, 41.9, 35.7, 28.3; MS (CI) 318, 267 (M⁺+1), 212, 168, 151 (100), 138].

Potassium carbonate and benzyl bromide were added to acetone having the dissolved the white crystalline compound and then the mixture was refluxed for 12 hours. After removing the solvent under reduced pressure, the mixture was extracted with water and ethyl acetate. The organic layer was dried with sodium sulfate and the sodium sulfate and the solvent were removed. A white solid was yielded by column chromatography (1.57 g, 95%) [¹H NMR (CDCl₃, 200 MHz) δ7.27-7.46 (m, 5H), 6.82 (d, J=8.0 Hz, 1H), 6.73 (d, J=1.8 Hz, 1H), 6.65 (dd, J=8.0, 1.8 Hz, 1H), 5.13 (s, 2H), 4.55 (br s, 1H), 3.88 (s, 3H), 3.44 (q, J=6.6 Hz, 2H), 2.72 (t, J=7.2 Hz, 2H), 1.44 (s, 9H); ¹³C NMR (CDCl₃, 50 MHz) δ155.9, 149.9, 147.0, 137.4, 132.4, 128.5, 127.7, 127.3, 120.8, 114.8, 112.9, 79.2, 71.4, 56.1, 42.0, 35.9, 28.4; LC/MS (ESP) 615, 559, 379, 358 (M⁺+1), 301 (100), 241, 224].

Trifluoroacetic acid (20 ml) was gently added to a dichloromethane solvent (20 ml) having the white solid (16.8 mmol, 6.0 g) at 0° C. After stirring the mixture for 40 minutes, the mixture solution was gently placed in a sodium bicarbonate solution with ice. The mixture was extracted with a diethylether solvent, which was then removed, and dissolved in chloroform to be neutralized with saturated sodium bicarbonate solution, and the solvent was removed. The resultant material was treated with 1M HCl in diethylether (20 ml) to give a white hydrochloride salt, intermediate 6 (1.32 g, 64%) [¹H NMR (CDCl₃, 200 MHz) δ8.24 (br s, 2H), 7.28-7.45 (m, 5H), 6.96 (d, J=8.0 Hz, 1H), 6.90 (d, J=1.8 Hz, 1H), 6.73 (dd, J=8.0, 1.8 Hz, 1H), 5.04 (s, 1H), 3.77 (s, 3H), 2.96-3.03 (m, 2H), 2.80-2.88 (m, 2H); ¹³C NMR (CDCl₃, 50 MHz) δ149.3, 146.6, 137.2, 130.3, 128.2, 127.6, 127.5, 120.5, 114.1, 113.0, 70.1, 55.6, 32.4; MS (CI): 258 (M⁺), 241 (100), 228, 91].

### \textcircled{2} General Synthesis Method and Analysis of N-[2-(4-benzyloxy-3-methoxyphenyl)ethyl]alkyl amides (7a-h)

Alkyl chlorides (propionyl, butyryl, isobutryl, -phenylacetyl, 4-methylbutyryl, cyclopropanecarbonyl, cyclobutanecarbonyl and cyclohexanecarbonyl chlorides) were added to a dichloromethane solvent having the dissolved compound 6, and triethyl amine was gently added thereto at 0° C. The mixture was stirred for 30 minutes to one hour. The solvent was removed under reduced pressure, and water was added. Organic substances in the resultant material were extracted with ethyl acetate. The organic layers were washed with water, dried with sodium sulfate, and then filtered. The solvent was removed from the filtered solution under reduced pressure and recrystallization or column chromatography yielded compounds 7a-h.

### N-[2-[(4-benzyloxy-3-methoxyphenyl)ethyl]propionamide (7a)

Compound 6 (3.50 mmol, 900 mg), propionyl chloride (4.55 mmol, 421 mg), triethylamine (10.5 mmol, 1.06 g), yield-7a (984 mg, 90%): ¹H NMR (CDCl₃, 200 MHz) δ7.26-7.46 (m, 5H), 6.81 (d, J=8.0 Hz, 1H), 6.73 (d, J=2.2 Hz, 1H), 6.64 (dd, J=8.2, 1.9 Hz, 1H), 5.65 (br s, 1H), 5.12 (s, 2H), 3.86 (s, 3H), 3.46 (q, J=6.6 Hz, 2H), 2.73 (t, J=7.0 Hz, 2H), 2.14 (q, J=7.5 Hz, 2H), 1.11 (t, J=7.7 Hz, 3H); ¹³C NMR (CDCl₃, 50 MHz) δ173.7, 149.6, 146.7, 137.1, 132.1, 128.4, 127.7, 127.2, 120.5, 114.2, 112.4, 71.0, 55.8, 40.5, 35.2, 29.6, 9.8; MS (EI): 313 (M⁺), 240, 149, 137, 91 (100), 65, 57, 30.

### N-[2-[(4-benzyloxy-3-methoxyphenyl)ethyl]butylamide (7b)

Compound 6 (3.50 mmol, 900 mg), butyryl chloride (4.20 mmol, 448 mg), triethylamine (10.5 mmol, 1.06 g), yield-7B (1.08 g, 94%): NMR (CDCl₃, 200 MHz) δ7.46-7.26 (m, 5H), 6.81 (d, J=8.0 Hz, 1H), 6.73 (d, J=1.8 Hz, 1H), 6.64 (dd, J=8.0, 1.8, 1H), 5.56 (s, 1H), 5.12 (s, 2H), 3.86 (s, 3H), 3.47 (q, J=6.6 Hz, 2H), 2.74 (t, J=7.0 Hz, 2H), 2.09 (t, J=7.6 Hz, 2H), 1.61 (sext, J=7.4 Hz, 2H), 0.91 (t, J=7.3 Hz, 3H); ¹³C NMR (CDCl₃, 50 MHz) δ172.9, 149.7, 146.7, 137.2, 132.0, 128.4, 127.7, 127.2, 120.5, 114.2, 112.4, 71.1, 55.9, 40.5, 38.6.35.3, 19.1, 13.7; MS (EI): 327 (M⁺), 240, 149, 137, 91 (100), 43.

### N-[2[(4-benzyloxy-3-methoxyphenyl)ethyl]isobutylamide (7c)

Compound 6 (3.50 mmol, 900 mg), isobutyryl chloride (4.20 mmol, 448 mg), triethylamine (10.5 mmol, 1.06 g), yield-7C (1.10 g, 96%): ¹H NMR (CDCl₃, 200 MHz) δ7.46-7.26 (m, 5H), 6.82 (d, J=8.2 Hz, 1H), 6.73 (d, J=1.8 Hz, 1H), 6.64 (dd, J=8.0, 1.4, 1H), 5.56 (br s, 1H), 5.13 (s, 2H), 3.87 (s, 3H), 3.47 (q, J=6.6 Hz, 2H), 2.74 (t, J=6.9 Hz, 2H), 2.28 (quin, J=7.1 Hz, 1H), 1.11 (d, J=7.0 Hz, 2H); ¹³C NMR (CDCl₃, 50 MHz): δ176.8, 149.7, 146.7, 137.1, 132.1, 128.4, 127.7, 127.2, 120.6, 114.2, 112.4, 71.1, 55.9, 40.4, 35.5, 35.2, 19.5; MS (EI): 327 (M⁺), 240, 149, 137, 91(100), 43.

### iv) N-[2-[(4-benzyloxy-3-methoxyphenyl)ethyl]-2-phenylacetamide (7d)

Compound 6 (3.42 mmol, 1.0 g), triethyl amine (10.3 mmol, 1.04 g), phenylacetyl chloride (3.76 mmol, 518 mg), yield-7D (802 mg, 63%): NMR (CDCl₃, 200 MHz) δ7.71-7.47 (m, 10H), 6.65 (d, J=8.2 Hz, 1H), 6.62 (d, J=1.8 Hz, 1H), 6.44 (dd, J=8.2, 2.2 Hz, 1H), 5.39 (br s, 1H), 5.12 (s, 2H), 3.82 (s, 3H), 3.51 (s, 2H), 3.42 (q, J=6.4 Hz, 2H), 2.65 (t, J=6.7 Hz, 2H); ¹³C NMR (CDCl₃, 100 MHz) δ170.8, 149.6, 146.7, 137.2, 134.7, 131.7, 129.4, 128.9, 128.5, 127.8, 127.2, 127.1, 120.5, 114.1, 112.2, 71.0, 55.9, 43.8, 40.6, 35.0; MS (EI): 375 (M⁺), 240, 149, 137, 91(100), 65.

### v) N-[2-[(4-benzyloxy-3-methoxyphenyl)ethyl]-3-methylbutylamide (7e)

Compound 6 (3.50 mmol, 900 mg), isovaleryl chloride (4.20 mmol, 506 g), triethyl amine (10.5 mmol, 1.06 g), yield-7E (1.11 g, 93%): ¹H NMR (CDCl₃, 200 MHz) δ7.46-7.25 (m, 5H), 6.82 (d, J=8.0 Hz, 1H), 6.73 (d, J=1.8 Hz, 1H), 6.63 (dd, J=8.2, 2.0, 1H), 5.51 (br s, 1H), 5.13 (s, 2H), 3.87 (s, 3H), 3.49 (q, J=6.7 Hz, 2H), 2.74 (t, J=6.9 Hz, 2H), 2.15-1.95 (m, 3H), 0.90 (d, J=6.6 Hz, 6H); ¹³C NMR (CDCl₃, 50 MHz) δ172.4, 149.7, 146.7, 137.1, 132.0, 128.5, 127.7, 127.2, 120.5, 114.2, 112.4, 71.1, 55.9, 46.1, 40.4, 35.3, 22.4; MS (EI): 341 (M⁺), 240, 149, 137, 91 (100), 65, 57, 30.

### vi) N-[2-[(4-benzyloxy-3-methoxyphenyl)ethyl]-2-cyclopropylacetamide (7f)

Compound 6 (3.89 mmol, 1.0 g), cyclopropanocarbonyl chloride (5.45 mmol, 589 mg), triethyl amine (11.7 mmol, 1.18 g), yield-7f (1.12 g, 88%); NMR (CDCl₃, 200 MHz) δ7.45-7.26 (m, 5H), 6.82 (d, J=8.0 Hz, 1H), 6.74 (d, J=1.8 Hz, 1H), 6.58 (dd, J=8.0, 1.8 Hz, 1H), 5.77 (br s, 1H), 5.13 (s, 2H), 3.87 (s, 3H), 3.48 (q, J=6.6 Hz, 2H), 2.74 (t, J=7.0 Hz, 2H), 1.85-1.19 (m, 1H), 0.95 (quin, J=3.8 Hz, 2H), 0.74-0.64 (m, 2H); ¹³C NMR (CDCl₃, 50 MHz) δ173.4, 149.6, 146.7, 137.2, 132.1, 128.4, 127.7, 127.2, 120.5, 114.2, 112.4, 71.1, 55.9, 40.8, 35.3, 14.6, 7.0; MS (ED: 325 (M⁺), 240, 149, 137, 91 (100), 69, 41.

### vii) N-[2-[(4-benzyloxy-3-methoxyphenyl)ethyl]-2-cyclobutylacetamide (7g)

Compound 6 (3.50 mmol, 900 mg), cyclobutanecarbonyl chloride (4.55 mmol, 539 mg), triethyl amine (10.5 mmol, 1.06 g), yield-7 g (737 mg, 79%): ¹H NMR (CDCl₃, 200 MHz) δ7.46-7.25 (m, 5H), 6.81 (d, J=8.0 Hz, 1H), 6.73 (d, J=1.8 Hz, 1H), 6.64 (dd, J=8.2, 2.2 Hz, 1H), 5.49 (br s, 1H), 5.13 (s, 2H), 3.87 (s, 3H), 3.47 (q, J=6.6 Hz, 2H), 2.92 (quin, J=8.5 Hz, 1H), 2.73 (t, J=7.0 Hz, 2H), 2.37-1.98 (m, 4H), 1.94-1.78 (m, 2H); ¹³C NMR (CDCl₃, 50 MHz) δ174.9, 149.7, 146.7, 137.2, 132.1, 128.4, 127.7, 127.2, 120.6, 114.2, 112.4, 71.1, 55.9, 40.4, 39.9, 35.2, 25.3, 18.1; MS (EI): 339 (M⁺), 240, 149, 137, 91 (100), 55.

### viii) N-[2-[(4-benzyloxy-3-methoxyphenyl)ethyl]-2-cyclohexylacetamide (7h)

Compound 6 (3.50 mmol, 900 mg), cyclohexanecarbonyl chloride (4.55 mmol, 667 mg), triethyl amine (10.5 mmol, 1.06 g), yield-7 h (1.09 g, 85%): ¹H NMR (CDCl₃, 400 MHz) δ7.41-7.23 (m, 5H), 6.79 (d, J=8.0 Hz, 1H), 6.67 (d, J=2.0 Hz, 1H), 6.61 (dd, J=8.0, 2.0 Hz 1H), 5.48 (br s, 1H), 5.09 (s, 2H), 3.83 (s, 3H), 3.43 (q, J=6.5 Hz, 2H), 2.70 (t, J=6.8 Hz, 2H), 2.00-1.93 (m, 1H), 1.78-1.71 (m, 4H), 1.63-1.59 (m, 1H), 1.40-1.30 (m, 2H), 1.25-1.12 (m, 3H); ¹³C NMR (CDCl₃, 100 MHz) δ176.0, 149.7, 146.7, 137.2, 132.1, 128.4, 127.7, 127.2, 120.6, 114.3, 112.5, 71.1, 55.9, 45.4, 40.3, 35.2, 29.6, 25.6; MS (EI): 367 (M⁺), 240, 149, 137, 91 (100), 83, 55.

### \textcircled{3} Preparation and Analysis of 1-alkyl-7-hydroxy-6-methoxy-1,2,3,4-tetrahydroisoquinolines (8a-h)

Phosphorus oxychloride (POCl₃) was added to anhydrous acetonitrile containing compounds 7a-h and refluxed for 2 to 5 hours. The solvent was removed under reduced pressure and the result was dried in vacuum. The dried compound was dissolved in methanol, and sodium borohydride (NaBH₄) was gently added thereto at 0° C. After stirring the mixture at RT for 24 hours, the mixture was filtered with silica gel and dried with sodium sulfate. Saturated sodium bicarbonate was poured and an organic substance was extracted with a dichloromethane solvent, dried with sodium sulfate, and removed under reduced pressure. The vacuum-dried organic substance was dissolved in methanol, HCl solution and 10% palladium-carbon were added thereto, and the mixture was stirred under hydrogen gas at RT for 12 hours. The resultant solution was filtered with cellite and the solvent was removed under reduced pressure. The column chromatography or recrystallization yielded compounds 8a-h, as hydrochloride salts.

### 1-ethyl-7-hydroxy-6-methoxy-1,2,3,4-tetrahydroisoquinoline (8a)

Compound 7a (3.0 mmol, 940 mg), POCl₃ (9.0 mmol, 1.38 g), NaBH₄ (15 mmol, 587 mg), 10% palladium charcoal (100 mg); yield-8a (128 mg, 18%): ¹H NMR (DMSO-d₆, 400 MHz) δ9.69 (br s, 1H), 9.07 (br s, 1H), 9.02 (s, 1H), 6.73 (s, 1H), 6.66 (s, 1H), 4.23 (br s, 1H), 3.74 (s, 3H), 3.16 (br s, 1H), 3.01-2.93 (m, 1H), 2.86-2.80 (m, 1H), 1.97-1.87 (m, 2H), 1.01 (t, J=3.7 Hz, 3H); ¹³C NMR (DMSO-d₆, 100 MHz) δ147.1, 145.2, 124.5, 122.5, 113.0, 112.0, 55.6, 55.0, 54.6, 26.2, 24.5, 9.8; LC MS: 208.2 (M⁺+1, 100).

### 7-hydroxy-6-methoxy-1-propyl-1,2,3,4-tetrahydroisoquinoline (8b)

Compound 7B (2.99 mmol, 980 mg), POCl₃ (8.98 mmol, 1.38 g), NaBH₄ (23.9 mmol, 905 mg), 10% palladium charcoal (100 mg); yield-8b (590 mg, 77%): ¹H NMR (DMSO-d₆, 400 MHz) δ9.80-9.20 (br s, 1H), 9.04 (br s, 1H), 6.72 (s, 1H), 6.66 (s, 1H), 4.26 (t, J=3.2 Hz, 1H), 3.74 (s, 3H), 3.36-3.30 (m, 1H), 3.00-2.93 (m, 1H), 2.85-2.78 (m, 1H), 1.88-1.81 (m, 2H), 1.52-1.42 (m, 2H), 0.92 (t, J=7.2 Hz, 3H); ¹³C NMR (DMSO-d₆, 100 MHz) δ147.1, 145.2, 124.9, 122.5, 123.0, 112.0, 55.6, 53.5, 35.6, 24.5, 18.2, 13.7; LC MS: 479.2, 222.2 (M⁺+1, 100).

### 7-hydroxy-1-isopropyl-6-methoxy-1,2,3,4-tetrahydroisoquinoline (8c)

Compound 7c (2.99 mmol, 980 mg), POCl₃ (8.98 mmol, 1.38 g), NaBH₄ (23.9 mmol, 905 mg), 10% palladium charcoal (100 mg); yield-8c (536 mg, 70%): ¹H NMR (DMSO-d₆, 400 MHz) δ9.81 (br s, 1H), 9.04 (br s, 1H), 8.66 (br s, 1H), 6.69 (s, 1H), 4.26 (br t, J=3.4, 1H), 3.75 (s, 3H), 3.37 (br s, 1H), 3.10-3.08 (m, 2H), 2.78-2.73 (m, 1H), 2.37-2.29 (m, 1H), 1.08 (d, J=7.2 Hz, 3H), 0.84 (d, J=7.2 Hz, 3H); ¹³C NMR (DMSO-d₆, 100 MHz) δ146.9, 145.3, 123.7, 123.4, 113.0, 111.9, 58.9, 55.5, 43.3, 30.8, 24.6, 19.2, 16.2; LC MS: 479.2, 222.2 (M⁺+1, 100).

### 1-benzyl-7-hydroxy-6-methoxy-1,2,3,4-tetrahydroisoquinoline (8d)

Compound 7d (0.51 mmol, 135 mg); yield-8d (101 mg, 74%): ¹H NMR (CDCl₃, 400 MHz) δ7.22-7.36 (m, 5H), 6.62 (s, 1H), 6.57 (s, 1H), 4.04-4.10 (m, 1H), 3.82 (s, 3H), 3.13-3.22 (m, 2H), 2.70-2.90 (m, 3H), 2.62-2.70 (m, 1H); ¹³C NMR (CDCl₃, 100 MHz) δ145.1, 143.6, 139.1, 131.2, 129.3, 128.6, 126.6, 126.4, 112.0, 111.1, 56.8, 55.9, 42.4, 40.9, 29.5; MS (CI): 270 (M⁺+1), 178 (100).

### 7-hydroxy-1-isobutyl-6-methoxy-1,2,3,4-tetrahydroisoquinoline (8e)

Compound 7e (3.13 mmol, 1.07 mg), POCl₃ (9.40 mmol, 1.44 g), NaBH₄ (25.0 mmol, 947 mg), 10% palladium charcoal (100 mg); yield-8e (567 mg, 67%): NMR (DMSO-d₆, 400 MHz) δ9.75 (br s, 1H), 9.36 (br s, 1H), 9.06 (s, 1H), 6.72 (s, 1H), 6.64 (s, 1H), 4.27 (br d, J=3.6 Hz, 1H), 3.74 (s, 3H), 3.37-3.11 (m, 1H), 3.20-3.08 (m, 1H), 3.02-2.90 (m, 1H), 2.90-2.78 (m, 1H), 2.00-1.89 (m, 1H), 1.88-1.77 (m, 1H), 1.65-1.55 (m, 1H), 0.98 (d, J=6.4 Hz, 3H), 0.95 (d, J=6.8 Hz, 3H); ¹³C NMR (DMSO-d₆, 100 MHz) δ147.0, 145.2, 125.4, 122.5, 113.0, 112.0, 55.6, 51.8, 43.3, 38.7, 24.4, 23.8, 23.0, 21.6; LC MS: 236.2 (M⁺+1, 100).

### 1-cyclopropyl-7-hydroxy-6-methoxy-1,2,3,4-tetrahydroisoquinoline (8f)

Compound 7f (3.07 mmol, 1.0 g), POCl₃ (9.22 mmol, 1.4 g), NaBH₄ (24.6 mmol, 929 mg), 10% palladium charcoal (100 mg); yield-8f (458 mg, 58%): ¹H NMR (DMSO-d₆, 400 MHz) δ9.64 (br s, 2H), 9.09 (s, 1H), 6.97 (s, 1H), 6.73 (s, 1H), 3.74 (s, 3H), 3.55 (d, J=9.6 Hz, 1H), 3.41-3.36 (m, 1H), 3.14-2.97 (m, 2H), 2.86-2.77 (m, 1H), 1.18-1.09 (m, 1H), 0.88-0.81 (m, 1H), 0.81-0.72 (m, 1H), 0.72-0.64 (m, 1H), 0.60-0.52 (m, 1H); ¹³C NMR (DMSO-d₆, 100 MHz) J 147.3, 145.2, 125.1, 122.4, 113.1, 111.9, 58.9, 55.6, 39.8, 24.5, 14.6, 5.8, 2.8; LC MS: 220.2 (M⁺+1, 100), 203.2.

### vii) 1-cyclobutyl-7-hydroxy-6-methoxy-1,2,3,4-tetrahydroisoquinoline (8g)

Compound 7g (2.95 mmol, 1.0 g), POCl₃ (8.8 mmol, 1.36 g), NaBH₄ (23.6 mmol, 893 mg), 10% palladium charcoal (100 mg); yield-8g (645 mg, 81%): ¹H NMR (DMSO-d₆, 400 MHz) δ9.90-9.00 (br s, 2H), 9.07 (s, 1H), 6.73 (s, 1H), 6.62 (s, 1H), 4.20 (d, J=9.2, one conformer of C1-H), 4.14 (br d, J=4.8, one conformer of C1-H), 3.73 (s, 3H), 3.31-3.25 (m, 1H), 3.16 (d, J=4.0 Hz, 1H), 3.13-3.07 (m, 1H), 2.98-2.91 (m, 1H), 2.85-2.76 (m, 1H), 2.78-2.67 (m, 1H), 2.17-2.02 (m, 3H), 2.02-1.92 (m, 1H), 1.89-1.70 (m, 2H); ¹³C NMR (DMSO-d₆, 100 MHz) δ147.1, 145.1, 124.1, 122.4, 112.9, 112.1, 57.5, 55.5, 48.6, 38.3, 26.8, 25.2, 24.5, 17.6; LC MS: 234.2 (M⁺+1, 100).

### 1-cyclohexyl-7-hydroxy-6-methoxy-1,2,3,4-tetrahydroisoquinoline (8h)

Compound 7h (2.53 mmol, 930 mg), POCl₃ (7.59 mmol, 1.16 g), NaBH₄ (20.2 mmol, 757 mg), 10% palladium charcoal (100 mg); yield-8h (541 mg, 72%): ¹H NMR (DMSO-d₆, 400 MHz) δ9.85 (br s, 1H), 9.03 (s, 1H), 8.67 (br s, 1H), 6.73 (s, 1H), 6.67 (s, 1H), 4.22 (br s, 1H), 3.74 (s, 3H), 3.53 (br s, 1H), 3.04 (br s, 1H), 2.98-2.90 (m, 1H), 2.80-2.74 (m, 1H), 1.91 (br s, 1H), 1.80-1.58 (m, 4H), 1.50-1.00 (m, 6H); ¹³C NMR (DMSO-d₆, 100 MHz) δ147.0, 145.1, 123.4, 123.3, 113.3, 112.0, 58.4, 55.5, 40.7, 29.3, 26.2, 25.9, 25.7, 25.6, 24.5; LC MS: 262.2 (M⁺+, 100).

### \textcircled{4} Synthesis Method and Analysis of 2-acetyl-1-alkyl-7-hydroxy-6-methoxy-1,2,3,4-tetrahydroisoquinolines (9a-h)

A dichloromethane solvent was added to a reaction container containing compounds 8a-h, and acetic anhydride and triethyl amine were sequentially added at RT. After stirring the mixture for about 2 hours, the solvent was removed under reduced pressure, and column chromatography yielded crude compounds 9a-h. After that, recrystallization yielded pure compounds 9a-h.

### i) 2-acetyl-1-ethyl-7-hydroxy-6-methoxy-1,2,3,4-tetrahydroisoquinoline (9a)

Compound 8a (85 mg, 0.36 mmol), acetic anhydride (36 mg, 0.35 mmol), tri-ethylamine (177 mg, 1.75 mmol); yield-9a (51 mg, 58%): ¹H NMR (CDCl₃, 400 MHz) δ6.70 (s, one conformer of C6-H or C8-H), 6.65 (s, one conformer of C6-H or C8-H), 6.58 (s, one conformer of C6-H or C8-H), 6.56 (s, one conformer of C6-H or C8-H), 5.46-5.40 (dd, J=8.8, 6.0 Hz, one conformer of C1-H), 4.66-4.59 (m, one conformer of C3-H), 4.59-4.53 (t, J=7.2 Hz, one conformer of C1-H), 3.859 (s, one conformer of OCH₃), 3.850 (s, one conformer of OCH₃), 3.78-3.73 (m, one conformer of C3-H), 3.55-3.42 (m, one conformer of C3-H), 3.06-2.97 (m, one conformer of C3-H), 2.94-2.79 (m, one conformer of C4-H, 1H), 2.79-2.70 (m, one conformer of C4-H), 2.65-2.57 (m, one conformer of C4-H), 2.17 (s, one conformer of —NCOCH), 2.16 (s, one conformer of —NCOCH₃), 1.88-1.70 (m, 2H), 0.96 (td, J=7.2, 29.2 Hz, 3H). Anal. (C₁₄H₁₉NO₃) calculated C, 67.45; 1-1, 7.68; N, 5.62; found C, 67.32; H, 7.84; N, 5.67.

### 2-acetyl-7-hydroxy-6-methoxy-1-propyl-1,2,3,4-tetrahydroisoquinoline (9b)

Compound 8b (200 mg, 0.78 mmol), acetic anhydride (79 mg, 0.78 mmol), triethylamine (236 mg, 2.34 mmol); yield-9b (92 mg, 45%): ¹H NMR (CDCl₃, 400 MHz) δ6.69 (s, one conformer of C6-H or C8-H), 6.64 (s, one conformer of C6-H or C8-H), 6.58 (s, one conformer of C6-H or C8-H), 6.56 (s, one conformer of C6-H or C8-H), 5.54-5.48 (m, one conformer of C1-H), 4.70-4.56 (m, one conformer of C1-H and C3-H), 3.854 (s, one conformer of OCH), 3.845 (s, one conformer of OCH₃), 3.79-3.71 (m, one conformer of C3-H), 3.57-3.47 (m, one conformer of C3-H), 3.10-3.00 (m, one conformer of C3-H), 2.95-2.58 (m, one conformer of C4-H, 2H), 2.164 (s, one conformer of —NCOCH₃), 2.157 (s, one conformer of —NCOCH₃), 1.88-1.60 (m, 2H), 1.50-1.24 (m, 2H), 1.12-0.84 (m, 3H); ¹³C NMR (CDCl₃, 100 MHz) δ169.4, 145.5, 144.1, 143.8, 130.8, 129.9, 125.5, 124.4, 113.3, 112.5, 110.9, 110.4, 57.1, 55.9, 51.9, 40.7, 29.4, 38.7, 35.4, 28.7, 27.6, 21.8, 21.7, 19.9, 19.6, 14.0. Anal. (C₁₅H₂₁NO₃) calculated C, 68.42; H, 8.04; N, 5.32; found C, 68.48; H, 8.04; N, 5.30.

### 2-acetyl-7-hydroxy-6-methoxy-1-isopropyl-1,2,3,4-tetrahydroisoquinoline (9c)

Compound 8c (200 mg, 0.78 mmol), acetic anhydride (79 mg, 0.78 mmol), tri-ethylamine (394 mg, 3.9 mmol); yield-9c (141 mg, 67%): ¹H NMR (CDCl₃, 400 MHz) δ6.73 (s, one conformer of C6-H or C8-H), 6.67 (s, one conformer of C6-H or C8-H), 6.61 (s, one conformer of C6-H or C8-H), 6.60 (s, one conformer of C6-H or C8-H), 5.18 (d, J=8.8 Hz, one conformer of C1-H), 4.54-4.44 (m, one conformer of C3-H), 4.16 (d, J=8.2 Hz, one conformer of C1-H), 3.857 (s, one conformer of OCH₃), 3.850 (s, one conformer of OCH₃), 3.74-3.62 (m, one conformer of C3-H), 3.26-3.16 (m, one conformer of C3-H), 2.95-2.72 (m, one conformer of C4-H, 2H), 2.15 (s, 3H), 2.08-1.90 (m, 1H), 1.14-0.92 (m, 6H); ¹³C NMR (CDCl₃, 100 MHz) δ169.9, 169.8, 145.7, 145.5, 143.5, 143.1, 130.0, 128.9, 125.6, 124.9, 114.6, 113.8, 111.0, 110.4, 63.6, 57.7, 55.9, 42.0, 36.5, 33.7, 33.4, 28.0, 26.8, 22.0, 21.98, 20.5, 20.2, 20.0, 19.8. Anal. (C₁₅H₂₁NO₃) calculated C, 68.42; H, 8.04; N, 5.32; found C, 68.17; H, 8.28; N, 5.35.

### 2-acetyl-1-benzyl-7-hydroxy-6-methoxy-1,2,3,4-tetrahydroisoquinoline (9d)

Compound 8d (69 mg, 0.26 mmol), acetic anhydride (29 mg, 0.28 mmol); yield-9d (75 mg, 94%): ¹H NMR (CDCl₃, 400 MHz) δ7.40-6.95 (m, 5H), 6.72 (s, one conformer of C6-H or C8-H), 6.58 (s, one conformer of C6-H or C8-H), 6.50 (s, one conformer of C6-H or C8-H), 6.48 (s, one conformer of C6-H or C8-H), 4.62-4.40 (m, one conformer of C1-H), 3.85 (s, one conformer of OCH₃), 3.82 (s, one conformer of OCH₃), 3.15-2.95 (m, one conformer of C3-H and one conformer of C4-H), 2.90-2.80 (m, one conformer of benzyl-H), 2.55-2.52 (m, one conformer of benzyl-H), 2.55-2.45 (m, one conformer of benzyl-H), 2.06 (s, one conformer of —NCOCH₃), 1.40 (s, one conformer of —NCOCH₃); MS (CI): 312 (M⁺+1, 100), 220, 178.

### 2-acetyl-7-hydroxy-6-methoxy-1-isobutyl-1,2,3,4-tetrahydroisoquinoline (9e)

Compound 8e (199 mg, 0.73 mmol), acetic anhydride (75 mg, 0.73 mmol), triethylamine (370 mg, 3.66 mmol); yield-9e (148 mg, 73%): ¹H NMR (CDCl₃, 400 MHz) δ6.66 (s, one conformer of C6-H or C8-H), 6.61 (s, one conformer of C6-H or C8-H), 6.58 (s, one conformer of C6-H or C8-H), 6.54 (s, one conformer of C6-H or C8-H), 5.64-5.56 (m, one conformer of C1-H), 4.70-4.64 (m, one conformer of C1-H), 4.58-4.39 (m, one conformer of C3-H), 3.86 (s, one conformer of OCH₃), 3.84 (s, one conformer of OCH₃), 3.80-3.71 (m, one conformer of C3-H), 3.58-3.47 (m, one conformer of C3-H), 3.18-3.07 (m, one conformer of C3-H), 2.94-2.81 (m, one conformer of C4-H, 1H), 2.76-2.69 (m, one conformer of C4-H, 1H), 2.17 (s, one conformer of —NCOCH₃), 2.157 (s, one conformer of —NCOCH₃), 1.83-1.38 (m, 3H), 1.10-0.88 (m, 6H); ¹³C NMR (CDCl₃, 100 MHz) δ169.8, 169.5, 145.5, 145.2, 144.1, 143.8, 131.1, 129.9, 125.5, 124.4, 113.3, 112.5, 110.9, 110.5, 55.9, 55.6, 50.4, 46.5, 46.0, 40.3, 35.8, 28.6, 27.4, 25.0, 24.7, 23.4, 23.1, 22.7, 22.4, 21.6, 21.4. Anal. (C₁₆H₂₃NO₃) calculated C, 69.29; H, 8.36; N, 5.05; found C, 69.26; H, 8.60; N, 5.02.

### 2-acetyl-1-cyclopropyl-7-hydroxy-6-methoxy-1,2,3,4-tetrahydroisoquinoline (9f)

Compound 8f (196 mg, 0.77 mmol), acetic anhydride (78 mg, 0.77 mmol), tri-ethylamine (389 mg, 3.9 mmol); yield-9f (145 mg, 72%): ¹H NMR (CDCl₃, 400 MHz) δ6.83 (s, one conformer of C6-H or C8-H), 6.74 (s, one conformer of C6-H or C8-H), 6.59 (s, one conformer of C6-H or C8-H), 6.56 (s, one conformer of C6-H or C8-H), 4.95 (d, J=8.8 Hz, one conformer of C1-H), 4.72-4.63 (m, one conformer of C3-H), 4.17 (d, J=8.0 Hz, one conformer of C1-H), 3.858 (s, one conformer of OCH₃), 3.848 (s, one conformer of OCH₃), 3.85-3.78 (m, one conformer of C3-H), 3.72-3.63 (m, one conformer of C3-H), 3.30-3.18 (m, one conformer of C3-H), 2.96-2.60 (m, one conformer of C4-H, 2H), 2.17 (s, one conformer of —NCOCH₃), 2.12 (s, one conformer of —NCOCH₃), 1.30-1.10 (m, 1H), 0.77-0.48 (m, 3H), 0.44-0.34 (m, 6H); ¹³C NMR (CDCl₃, 100 MHz) δ169.0, 145.5, 144.0, 143.7, 129.9, 128.5, 125.7, 124.5, 114.6, 113.3, 112.7, 110.8, 110.4, 60.4, 55.9, 55.5, 41.2, 36.1, 28.8, 27.7, 21.7, 21.5, 18.0, 17.8, 5.31, 5.25, 2.9, 2.5. Anal. (C₁₅H₁₉NO₃) calculated C, 68.94; H, 7.33; N, 5.36; found C, 68.94; H, 7.47; N, 5.35.

### 2-acetyl-1-cyclobutyl-7-hydroxy-6-methoxy-1,2,3,4-tetrahydroisoquinoline (9 g)

Compound 8g (237 mg, 0.88 mmol), acetic anhydride (89 mg, 0.88 mmol), tri-ethylamine (444 mg, 4.4 mmol); yield-9g (195 mg, 80%): NMR (CDCl₃, 400 MHz) δ6.70 (s, one conformer of C6-H or C8-H), 6.65 (s, one conformer of C6-H or C8-H), 6.58 (s, one conformer of C6-H or C8-H), 6.56 (s, one conformer of C6-H or C8-H), 5.46 (d, J=9.6 Hz, one conformer of C1-H), 4.68-4.60 (m, one conformer of C3-H), 4.50 (d, J=9.2 Hz, one conformer of C1-H), 3.85 (s, one conformer of OCH₃), 3.84 (s, one conformer of OCH₃), 3.77-3.69 (m, one conformer of C3-H), 3.58-3.44 (m, one conformer of C3-H), 3.04-2.95 (m, one conformer of C3-H), 2.92-2.68 (m, one conformer of C4-H, 2H), 2.66-2.55 (m, 1H), 2.21 (s, one conformer of —NCOCH₃), 2.15 (s, one conformer of —NCOCH₃), 2.10-1.90 (m, 3H), 1.89-1.68 (m, 3H); ¹³C NMR (CDCl₃, 100 MHz) δ169.4, 169.3, 145.6, 145.4, 143.8, 143.5, 129.5, 128.4, 125.2, 124.1, 113.0, 112.2, 111.1, 110.6, 61.5, 56.0, 55.9, 41.4, 41.1, 41.0, 35.7, 28.6, 27.8, 27.6, 27.4, 26.1, 25.5, 21.9, 21.7, 17.6. Anal. (C₁₆H₂₁NO₃) calculated C, 69.79; H, 7.69; N, 5.09; found C, 70.01; H, 7.81; N, 5.06.

### 2-acetyl-1-cyclohexyl-7-hydroxy-6-methoxy-1,2,3,4-tetrahydroisoquinoline (9h)

Compound 8h (200 mg, 0.67 mmol), acetic anhydride (68 mg, 0.67 mmol), tri-ethylamine (339 mg, 3.36 mmol); yield-9h (162 mg, 80%): ¹H NMR (CDCl₃, 400 MHz) δ6.72 (s, one conformer of C6-H or C8-H), 6.63 (s, one conformer of C6-H or C8-H), 6.61 (s, one conformer of C6-H or C8-H), 6.60 (s, one conformer of C6-H or C8-H), 5.19 (d, J=8.8 Hz, one conformer of C1-H), 4.52-4.43 (m, one conformer of C3-H), 4.29 (d, J=9.6 Hz, one conformer of C1-H), 3.86 (s, one conformer of OCH₃), 3.85 (s, one conformer of OCH₃), 3.70-3.60 (m, one conformer of C3-H), 3.25-3.16 (m, one conformer of C3-H), 2.96-2.73 (m, one conformer of C4-H, 2H), 2.15 (s, one conformer of —NCOCH₃), 2.14 (s, one conformer of —NCOCH₃), 1.84-1.50 (m, 6H), 1.22-0.94 (m, 5H); ¹³C NMR (CDCl₃, 100 MHz) δ169.98, 169.92, 145.7, 145.5, 143.4, 143.0, 129.5, 128.6, 125.6, 124.8, 114.8, 114.0, 111.0, 110.4, 62.8, 57.1, 55.9, 42.9, 42.6, 42.1, 36.5, 31.0, 30.6, 30.2, 29.8, 27.9, 26.8, 26.4, 26.2, 26.16, 26.1, 26.06, 22.0, 21.9. Anal. (C₁₈H₂₅NO₃) calculated C, 71.26; H, 8.31; N, 4.62; found C, 70.93; H, 8.53; N, 4.62.

### Exemplary Embodiment 3

### Synthesis Method of 7-hydroxy-6-methoxy-1,2,3,4-tetrahydroisoquinoline (HMTIO) Derivatives (11a-e and 12a-f) Substituted with Amides and Alkyls in N2 Position and Analysis of their Structures

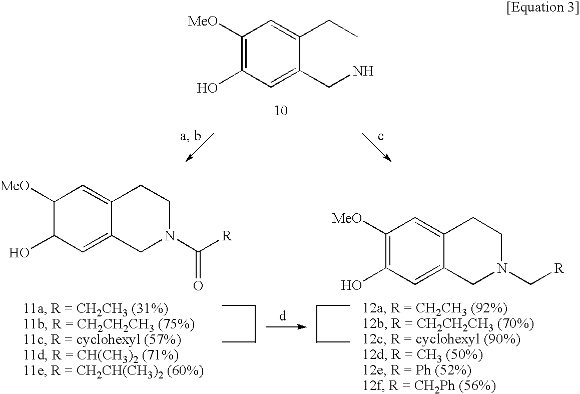

### \textcircled{1} Preparation and Analysis of HMTIO Derivatives (11a-e) Substituted with Amides in N2 Position

Preparations a) and b) Compound 10 (1.0 or 2.0 mmol) was dissolved in a dichloromethane solvent (10-15 ml), and alkylacyl chloride (propionic anhydride, butyryl chloride, cyclohexanecarbonyl chloride, isobutyryl chloride or 3-methylbutyryl chloride) was gently added thereto. Triethylamine (3.0 or 6.0 mmol) was gently added and the mixture was stirred at RT for about one hour. The reaction was quenched with water and the organic layer was washed with water. The solvent was removed under reduced pressure. The resultant compound was dissolved in methanol (10-20 ml) and calcium carbonate (3.0 or 6.0 mmol) was added thereto, followed by refluxing of the mixture for about 2 to 3 hours. The refluxed solution was filtered and then extracted with abundant dichloromethane solvent, and the organic layer was washed with 1.0M HCl solution and water. The solvent was removed under reduced pressure, and column chromatography yielded HMTIQ derivatives (11a-e) substituted with amides in N2 position.

### 2-ethylcarbonyl-7-hydroxy-6-methoxy-1,2,3,4-tetrahydroisoquinoline (EHMTIQ, 11a)

Compound 10 (420 Mg, 2.0 Mmol), Propionic Anhydride (410 Mg, 3.0 Mmol), Tri-ethylamine (1.0 [symbol] ), calcium carbonate (370 mg); yield-11a (146 mg, 31%):¹H NMR (CDCl₃, 200 MHz) δ6.71 (s, one conformer of C5-H or C8-H), 6.66 (s, one conformer of C5-H or C8-H), 6.62 (s, one conformer of C5-H or C8-H), 6.60 (s, one conformer of C5-H or C8-H), 6.10 (s, one conformer of O—H), 5.96 (s, one conformer of O—H), 4.62 (s, one conformer of C1-H), 4.50 (s, one conformer of C1-H), 3.86 (s, O—CH₃, 3H), 3.95-3.49 (m, C4-H, 2H), 2.85-2.70 (m, C3-H, 2H), 2.46 (q, J=7.4 Hz, 2H), 1.15-1.05 (m, 3H); ¹³C NMR (CDCl₃, 50 MHz) δ172.8, 145.5, 144.6, 144.3, 126.12, 126.11, 112.5, 111.8, 111.0, 110.6, 56.0, 46.8, 43.8, 43.2, 39.8, 29.1, 28.1, 26.9, 26.7, 9.4; MS (EI) 235 (M⁺, 100), 220, 178, 163, 150, 135.

### 7-hydroxy-6-methoxy-2-propylcarbonyl-1,2,3,4-tetrahydroisoquinoline (11b)

Compound 10 (215 mg, 1.0 mmol), butyryl chloride (93 mg, 1.0 mmol), triethylamine (0.45 ml), calcium carbonate (100 mg); yield-11b (187 mg, 75%): ¹H NMR (CDCl₃, 100 MHz) δ6.71 (s, one conformer of C5-H or C8-H), 6.66 (s, one conformer of C5-H or C8-H), 6.61 (s, one conformer of C5-H or C8-H), 6.60 (s, one conformer of C5-H or C8-H), 6.21 (s, one conformer of O—H), 6.03 (s, one conformer of O—H), 4.62 (s, one conformer of C1-H), 4.50 (s, one conformer of C1-H), 3.85 (s, O—CH₃, 3H), 3.95-3.60 (m, C4-H, 2H), 2.90-2.70 (m, C3-H, 2H), 2.46 (t, J=7.4 Hz, 2H), 1.80-1.60 (m, 2H), 1.05-0.90 (m, 3H); ¹³C NMR (CDCl₃, 50 MHz) δ172.1, 145.8, 144.7, 144.5, 126.2, 125.2, 112.7, 111.9, 111.2, 110.8, 56.1, 47.1, 43.9, 43.5, 39.9, 35.8, 35.7, 29.3, 28.2, 18.7, 14.1; MS (EI) 249 (M⁺, 100), 220, 178, 163, 150.

### 2-cyclohexylcarbonyl-7-hydroxy-6-methoxy-1,2,3,4-tetrahydroisoquinoline (11c)

Compound 10 (430 mg, 2.0 mmol), cyclohexanecarbonyl chloride (590 mg, 4.0 mmol), triethylamine (0.90 [symbol] ), calcium carbonate (320 mg); yield-11c (302 mg, 57%): ¹H NMR (CDCl₃, 200 MHz) δ6.72 (s, one conformer of C5-H or C8-H), 6.68 (s, one conformer of C5-H or C8-H), 6.60 (s, one conformer of C5-H or C8-H), 5.95 (s, one conformer of O—H), 5.76 (s, one conformer of O—H), 4.61 (s, one conformer of C1-H), 4.54 (s, one conformer of C1-H), 3.86 (s, O—CH₃, 3H), 3.72 (m, C4-H, 2H), 3.82-3.62 (m, 2H), 2.85-2.64 (m, C3-H, 2H), 2.62-2.45 (m, 1H), 1.90-1.20 (m, 10H); MS (EI) 289 (M⁺, 100), 274, 178, 163, 150.

### 7-hydroxy-6-methoxy-2-isopropylcarbonyl-1,2,3,4-tetrahydroisoquinoline (11d)

Compound 10 (215 mg, 1.0 mmol), isobutyryl chloride (0.2117, 2.0 mmol), triethylamine (0.5 [symbol] ), calcium carbonate (160 mg); yield-11d (178 mg, 71%): ¹H NMR (CDCl₃, 200 MHz) δ6.76 (s, one conformer of C5-H or C8-H), 6.67 (s, one conformer of C5-H or C8-H), 6.61 (s, one conformer of C5-H or C8-H), 6.20 (s, one conformer of O—H), 6.00 (s, one conformer of O—H), 4.61 (s, one conformer of C1-H), 4.55 (s, one conformer of C1-H), 3.86 (s, O—CH₃, 3H), 3.95-3.60 (m, C4-H, 2H), 3.00-2.65 (m, 3H), 1.45 (t, J=6.6 Hz, 6H); ¹³C NMR (CDCl₃, 50 MHz) δ175.8, 145.5, 144.4, 126.3, 125.0, 112.6, 111.7, 111.0, 110.7, 56.0, 46.8, 44.0, 43.1, 40.1, 30.4, 29.4, 28.0, 19.3; MS (EI) 249 (M⁺, 100), 234, 206, 178, 163, 150.

### 7-hydroxy-6-methoxy-2-isobutylcarbonyl-1,2,3,4-tetrahydroisoquinoline (11e)

Compound 10 (215 mg, 1.0 mmol), isovaleryl chloride (0.25 [symbol] , 2.0 mmol), tri-ethylamine (0.5 ml), calcium carbonate (160 mg); yield-11e (158 mg, 60%): NMR (CDCl₃, 200 MHz) δ6.71 (s, one conformer of C5-H or C8-H), 6.66 (s, one conformer of C5-H or C8-H), 6.61 (s, one conformer of C5-H or C8-H), 6.60 (s, one conformer of C5-H or C8-H), 6.21 (s, one conformer of O—H), 6.04 (s, one conformer of O—H), 4.63 (s, one conformer of C1-H), 4.51 (s, one conformer of C1-H), 3.85 (s, O—CH₃, 3H), 3.95-3.60 (m, C4-H, 2H), 2.85-2.67 (m, 2H), 2.38-2.05 (m, 3H), 1.10-0.95 (m, 6H); ¹³C NMR (CDCl₃, 50 MHz) δ171.4, 145.7, 145.5, 144.6, 144.3, 126.2, 126.1, 125.0, 112.5, 111.7, 111.0, 110.6, 55.9, 47.2, 43.8, 43.6, 42.5, 42.3, 39.8, 29.2, 28.1, 25.6, 22.7; MS (EI) 263 (M⁺) 220 (100), 206, 178, 163, 150.

### \textcircled{2} Preparation of HMTIQ Derivatives Substituted with Alkyls in N2 Position (12a-f) and Analysis of their Structures

Preparation d) 1M lithium aluminum hydride (tetrahydrofuran) solution was added to distilled tetrahydrofuran solution (10-20 ml) in which the compounds 11a-c were dissolved, and the mixture was refluxed for 4 to 5 hours. The reaction was quenched and then about 5 equivalent weights of ethyl acetate, 1.0M potassium hydroxide solution, and water were added to the mixture. The resultant solution was extracted with a dichloromethane solvent and then column chromatography yielded compounds 12a-c.

Preparation c) The compound 10 was mixed with aldehyde (acetaldehyde, phenylacetaldehyde or benzaldehyde) and titanium isopropyl oxide and stirred for about 1 hour. The mixture was dissolved in ethanol and stirred with sodium cyanoborohydride at RT for about 20 hours, and the reaction was quenched with water. The resultant solution was filtered and the solvent was removed under reduced pressure. The resulting compound was purified by column chromatography and then dissolved in methanol. The addition of 35% HCl solution gave hydrochloride salts and recrystallization in diethylether yielded compounds 12d-f.

### 7-hydroxy-6-methoxy-2-propyl-1,2,3,4-tetrahydroisoquinoline, hydrochloride (12a)

According to preparation d): compound 11a (160 mg, 0.68 mmol), 1M lithium aluminum hydride (tetrahydrofuran) (0.6 [symbol] , 0.6 mmol); yield-12a (138 mg, 92%):¹H NMR (DMSO-d₆, 400 MHz) δ9.14 (bs, OH, 1H), 6.74 (s, 1H), 6.59 (s, 1H), 4.27 (d, J=12.6 Hz, 1H), 4.06 (dd, J=16.0, 8.0 Hz, 1H), 3.75 (s, 3H), 3.37 (bs, 1H), 3.25-3.00 (m, 4H), 2.95-2.90 (bm, 1H), 1.85-1.75 (m, 2H), 0.90 (t, J=7.2 Hz, 3H); ¹³C NMR (DMSO-d₆, 100 MHz) δ147.3, 145.4, 121.6, 120.1, 112.9, 111.8, 56.4, 55.6, 51.0, 48.7, 24.3, 16.8, 11.0; MS (EI) 221 (M⁺) 192 (100), 150.

### 2-butyl-7-hydroxy-6-methoxy-1,2,3,4-tetrahydroisoquinoline,hydrochloride (12b)

According to method d): compound 11b (140 mg, 0.56 mmol), 1M lithium aluminum hydride (tetrahydrofuran) (0.6 [symbol] , 0.6 mmol); yield-12b (92 mg, 70%):¹H NMR (DMSO-d₆, 200 MHz) δ6.77 (s, 1H), 6.60 (s, 1H), 4.34 (d, J=13.6 Hz, 1H), 4.06 (dd, J=15.2, 8.0 Hz, 1H), 3.75 (s, 3H), 3.37 (bs, 1H), 3.25-3.00 (m, 4H), 2.95-2.90 (bm, 1H), 1.85-1.75 (m, 2H), 1.50-1.25 (m, 2H), 0.93 (t, J=7.0 Hz, 3H); ¹³C NMR (DMSO-d₆, 50 MHz) δ147.3, 145.4, 121.6, 120.1, 112.9, 111.8, 55.6, 54.6, 51.0, 48.7, 25.1, 24.3, 19.5, 13.5; MS (EI) 235 (M) 192 (100), 150.

### 2-(cyclohexylmethyl)-7-hydroxy-6-methoxy-1,2,3,4-tetrahydroisoquinoline, hydrochloride (12c)

According to preparation d): compound 11c (175 mg, 0.6 mmol), 1M lithium aluminium hydride (tetrahydrofuran) (0.4 [symbol] , 0.4 mmol); yield-12c (149 mg, 90%):¹H NMR (DMSO-d₆, 200 MHz) δ6.76 (s, 1H), 6.61 (s, 1H), 4.34 (d, J=13.2 Hz, 1H), 4.11 (dd, J=15.2, 7.2 Hz, 1H), 3.74 (s, 3H), 3.65-2.65 (m, 6H), 2.00-1.65 (m, 6H), 1.40-1.80 (m, 5H); ¹³C NMR (DMSO-d₆, 50 MHz) δ147.3, 145.4, 121.5, 119.9, 113.1, 111.8, 60.6, 55.9, 55.6, 32.1, 30.7, 25.4, 25.0, 23.9, 18.5; MS (EI) 275 (M) 192 (100), 150.

### 2-ethyl-7-hydroxy-6-methoxy-1,2,3,4-tetrahydroisoquinoline, hydrochloride (12d)

Compound 10 (215 mg, 1.0 mmol), acetaldehyde (0.12 [symbol] , 2.0 mmol), titanium iso-propyloxide (370 mg, 1.3 mmol), 1.0M sodium cyanoborohydride solution (tetrahydrofuran) (0.6 ml, 0.6 mmol); yield-12d (103 mg, 50%): NMR (DMSO-d₆, 200 MHz) δ6.76 (s, 1H), 6.59 (s, 1H), 4.40-4.20 (bm, 1H), 4.20-3.95 (bm, 1H), 3.74 (s, 3H), 3.60-2.65 (m, 6H), 1.31 (t, J=7.6 Hz, 3H).

### 2-benzyl-7-hydroxy-6-methoxy-1,2,3,4-tetrahydroisoquinoline, hydrochloride (12e)

Compound 10 (215 mg, 1.0 mmol), benzaldehyde (0.1 ml, 1.0 mmol), titanium iso-propyloxide (370 mg, 1.3 mmol), 1.0M sodium cyanoborohydride solution (tetrahydrofuran) (0.6 ml, 0.6 mmol); yield-12e (140 mg, 52%).

### 7-hydroxy-6-methoxy-2-(2-phenylethyl)-1,2,3,4-tetrahydroisoquinoline, hydrochloride (12f)

Compound 10 (215 mg, 1.0 mmol), benzaldehyde (1.20, 1.0 mmol), titanium iso-propyloxide (370 mg, 1.3 mmol), 1M sodium cyanoborohydride solution (tetrahydrofuran) (0.6 [symbol] , 0.6 mmol); yield-12f (158 mg, 56%):¹H NMR (DMSO-d, 200 MHz) δ7.42-7.20 (m, 5H), 6.76 (s, 1H), 6.62 (s, 1H), 4.41 (d, J=14.8 Hz, 1H), 4.17 (dd, J=14.8, 7.4 Hz, 1H), 3.74 (s, 3H), 3.67 (bs, 1H), 3.48-3.05 (m, 6H), 3.30-2.80 (m, 2H); ¹³C NMR (DMSO-d₆, 50 MHz) δ147.4, 145.5, 137.2, 128.64, 128.61, 126.7, 121.6, 120.1, 112.9, 111.9, 55.8, 55.6, 51.0, 48.9, 29.4, 24.4.

Dichloromethane, ethyl acetate and hexane simply distilled with boiling stones were used as solvents, and a nameless solvent having at least 99% purity was purchased and used. Subsequently, TLC was viewed in the UV range or by a phosphomolybdate indicator. ¹H and ¹³C NMR spectra were recorded at 200 or 400 MHz on a Varian spectrometer. Chemical shifts were given in ppm and referenced to the internal solvent peak. A mass spectrum was recorded using a mass spectrometer provided by Inha University or Korean Basic Science Institute (KRISS) to determine the structure of the unknown compound.

### MODE FOR THE INVENTION

### Cell Culture

BV-2 microglial cell line, CATH.a neuron line and SK-N-BE(2)C neuron line were cultured in Dulbecco's modified Eagle's medium (DMEM) supplemented with 10% bovine serum, 100 IU/l penicillin, and 10 [symbol] /ml streptomycin at 37° C. in an atmosphere of 5% CO₂ and 95% air. The cells were planted on a polystyrene petri dish at the following densities: BV-2 (2.5×10⁵ cells/24 well or 2.6×10⁶ cells/60 mm dish; SK− N-BE(2)C (1.5×10⁵ cells/24 well); and CATH.a (2.4×10⁴ cells/96 well).

Measurement of NO Production

200 [symbol] of cell culture medium fraction and 100 [symbol] of Griess reagent (2.5% ₃ [symbol] HO₄, 1% sulfanilamide and 0.1% naphthylethylenediamine dihydrochloride) were mixed in a 96-well microtiter plate, and the absorbance of a sample was read at 540 nm using a microtiter plate reader (multi-well spectrophotometer). The concentration of nitrite was calculated using a standard curve for sodium nitrite.

Assay for NF-kB p65 Shift to Nucleus

The cells were washed with cold phosphate buffered saline (PBS) and gently suspended in 400 [symbol] buffer solution containing 10 mM HEPES (pH 7.9), 10 mM KCl, 0.1 mM EDTA, 0.1 mM EGTA, 1 mM DTT and 0.5 mM PMSF. The cell suspension was placed on ice for 15 minutes, and reacted with 25 [symbol] NP-40 (0.5%) for 10 seconds. Cen-trifugation for 30 seconds yielded nuclear pellets, which were then resuspended in 50 [symbol] of cold PBS containing 20 mM HEPES (pH 7.9), 400 mM NaCl, and 1 mM each of DTT, EDTA, EGTA and PMSF. The suspension was vortexted for 15 minutes. The nuclear extract was centrifuged at 11,000×g for 15 minutes to get supernatant solution, whose protein content was measured. Equal amounts of the cell extract (5 [symbol] ) were subjected to electrophoresis in a 10% SDS-polyacrylamide gel and then transferred onto a polyvinylidene difluoride-nitrocellulose membrane. The membrane was blocked with TBST containing 8% skim milk at RT for one hour, incubated with primary antibody, anti-NF-kB p65 antibody (1:500 dilution), at 4° C. overnight, and further incubated with secondary antibody conjugated with horseradish peroxidase for one more hour. Protein bands were detected by a chemiluminescence detection method according to the manufacturer's indication.

RT-PCR for GTPCH, iNOS, TNF-α, IL-1β and COX-2

5 [symbol] each of total RNA samples isolated from BV-2 cells were subjected to reverse-transcription (RT), and then polymerase chain reaction (PCR) for 30 cycles under the conditions of 94° C. for 30 seconds, 60° C. for 40 seconds and 72° C. for one minute. Primers used in the PCR were as follows: iNOS (forward, ATGTCCGAAG-CAAACATCAC; reverse, TAATGTC CAGGAAGTAGGTG), TNF-α (forward, CA-GACCCTCACACTCAGATCATCTT reverse, CAGAGCAATGACTC-CAAAGTAGACCT), IL-1β (forward, ATGGCAACTGTTCCTGAACTCAACT; reverse, CAGGACAGGTAT AGATTCTTTCCTTT), COX-2 (forward, CAGCAAATCCTTGCTGTTCC; reverse, TGGGCAAAGAATGCAAACATC), GTPCH (forward, GGATACCAGGAGACCAT CTCA; reverse, TAGCATGGTGC-TAGTGACAGT). RT-PCR for B2M was simultaneously performed as internal control. The PCR products were subjected to electrophoresis in a 1.5% agarose gel, thereby confirming the presence of a desired size of single band.

Measurement of Lactate Dehydrogenase (LDH) Activity

0.26 mM NADH, 2.87 mM sodium pyruvate and 100 mM potassium phosphate buffer (pH 7.4) were added to cell culture medium (50 [symbol] ) to make a total volume of 200 [symbol] , and then cultured at RT. The resultant NAD⁺ was measured at 340 nm for 5 minutes at 2-second intervals using a microplate spectrophotometer (SPECTRA MAX 340 pc; Molecular Devices, Menlo Park, Calif., USA).

Evaluation of Protective Effect Against Neuron Death Due to Substances, Released from Activated Microglial Cell

BV-2 microglial cells were planted in a 24-well Petri dish at a density of 2.5×10⁵ cells/ml. After an overnight culture, the cells were treated with 1 mg/ml lipopolysaccharide and EHMTIQ (11a) and then cultured for 12 more hours. At the same time, SK-N-BE(2)C cells were planted in a 24-well Petri dish at 0.5×10⁵ cells/ml and cultured for 24 hours. The culture medium for SK-N-BE(2) C cells was removed and the culture medium for BV-2 was added thereto. After 24 hours, the death rate of SK-N-BE(2)C cells was measured using LDH.

Measurement of Superoxide Production

BV-2 microglial cells were planted in a 96-well Petri dish at 0.5×10⁵ cells/ml. After a 24-hour culture, the cells were washed twice with Hank's balanced salt solution (HBSS) without phenol red and treated with EHMTIQ (11a) and WST-1. However, some samples were not treated with 20 [symbol] superoxide dismutase (SOD; 800 UI/ml). All samples were incubaed at 37° C. for 10 minutes. The absorbance of a sample was read at 450 nm using a SpectraMax Plus microplate spectrophotometer. The yield of superoxide was calculated according to difference in absorbance value between the samples with and without SOD.

Measurement of Free-Radical Scavenging Activity

Antioxidant activity was evaluated based on scavenging activity of 2,2-diphenyl-1-picrylhydrazyl (DPPH) radical. DPPH was dissolved in 80% methanol to make a final concentration of 100 [symbol] . 8 [symbol] of EHMTIQ (11a) dissolved in dimethyl sulfoxide reacted with 232 [symbol] of DPPH radical solution. The reaction mixture was incubated for 25 minutes at RT, and then the absorbance of DPPH was measured at 517 nm using a SpectraMax GEMINI XS fluorescence spectrophotometer (Molecular Devices, Sunnyvale, Calif., USA).

Evaluation for Drug Stability

1 mM EHMTIQ (11a) was added to 1 mg samples of white rat liver microsomes, and the samples were incubated at 37° C. for 0, 30, 120 and 240 minutes in the presence of a NADPH-regeneration system (2.6 mM β-NADP⁺, 10 mM glucose-6-phosphate, 4 UI/ml glucose-6-phosphate dehydrogenase and 10 mM MgCl₂). Perchloric acid was added to the sample to make a final concentration of 500 mM and then the reaction was stopped. The reaction mixture was centrifuged at 16,000×g for 20 minutes. The supernatant solution (120 l) was purified using a Waters HPLC system [717 plus autosampler, 515 pump, and Symmetry C18 column (4.6 mm×150 mm, 5 mm)] using a 5-30% linear gradient of acetonitrile as mobile phase. EHMTIQ (11a) was detected at 254 nm using a Waters 486 UV detector and analyzed using EMPOWER software (Millipore Corporation, Milford, Mass., USA).

Data Analysis

Data obtained from repeated tests were used to calculate a mean value±SEM. The data calculations were performed by analysis of variance (one-way ANOVA) with post Dunnett's multiple comparison test for comparison of the results with at least three controls. All statistical assays were preformed with PRISM (GraphPad Software, San Diego, Calif.). A p value of <0.05 was considered significant.

### Experimental Example 1

### Assays for Effects of EHMTIQ (11a)

Inhibitory Effect on NO Production

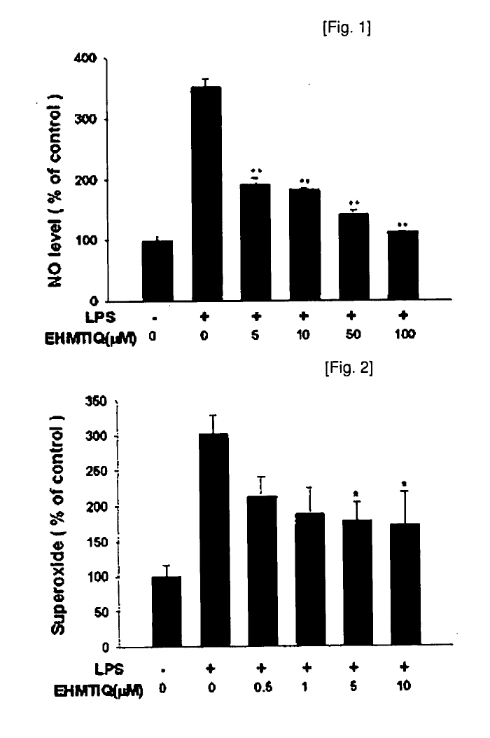

To determine whether EHMTIQ (11a) inhibits NO production in an activated microbial cell, a mouse microglial cell line, BV-2, was used in this assay. Lipopolysaccharide (LPS)-stimulated BV-2 cell samples were treated with various concentrations of EHMTIQ (11a) and then the NO level of a sample was measured. The results are illustrated in FIG. 1.

As seen from FIG. 1, LPS induced a significant increase (3.5±0.1 times) in NO level of the BV-2 cell. Here, when the cells were treated with EHMTIQ (11a), the NO level was inversely proportional to the concentration of EHMTIQ (11a). That is, a low concentration (5 [symbol] ) of EHMTIQ (11a) may decrease NO production induced by LPS to 63±4%, compared with that of the control not treated with EHMTIQ. And, a high concentration (100 [symbol] ) of EHMTIQ (11a) may inhibit NO production to the control level. Single treatment of EHMTIQ (11a) did not exhibit any cytotoxicity (not illustrated). The IC₅₀ value for EHMTIQ (11a) was determined to be 2.81 [symbol] .

Effect on Production of NADPH Oxidase-Derived Superoxide

The activation of a microglial cell led to the activation of NADPH oxidase and the production of superoxide. Accordingly, the assay was performed to determine whether EHMTIQ (11a) affects production of NADPH oxidase-derived superoxide. The LPS-stimulated BV-2 cell samples were treated with various concentrations of EHMTIQ (11a) and then the level of released superoxide was measured. The results are illustrated in FIG. 2.

Referring to FIG. 2, LPS induced an increase in superoxide production of 3.0±0.3 times compared with that of the non-activated cell. Here, the superoxide production was inversely proportional to the concentration of EHMTIQ (11a). 5 [symbol] or 10 [symbol] EHMTIQ (11a) may lower the superoxide production to 62±3.1% or 65±3.1%.

3) Effect on Expression of TNF-α Genes

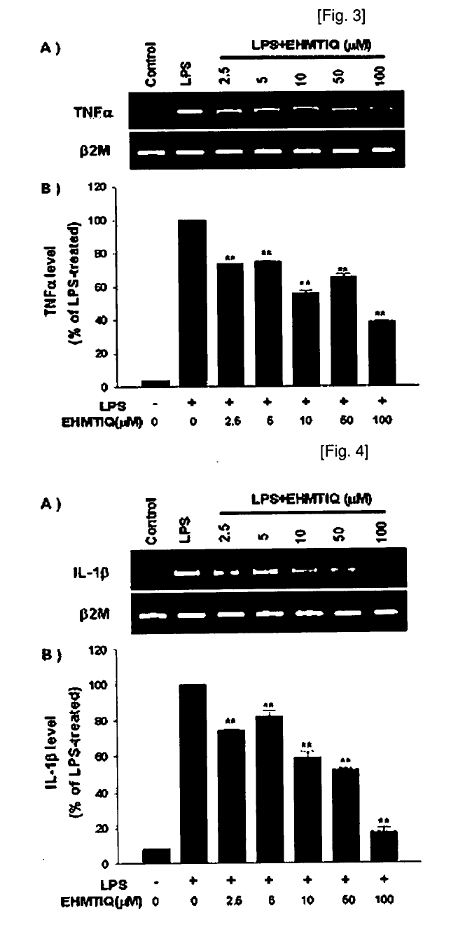

The assay was performed to determine whether EHMTIQ (11a) affects TNF-α production in an activated microglial cell. LPS-stimulated BV-2 cell samples were treated with various concentrations of EHMTIQ (11a) and the expression of TNF-α genes was estimated by RT-PCR. The results are illustrated in FIG. 3.

As seen from FIG. 3, LPS induced a significant increase in mRNA level of TNF-α (26±1 times), which was inversely proportional to the concentration of EHMTIQ (11a). A low concentration (2.5 [symbol] ) of EHMTIQ (11a) decreased the mRNA level to a statistically significant level, and particularly, 5 and 100 [symbol] EHMTIQ (11a) decreased the mRNA level of TNF-α to 74±1% and 36±1%, respectively compared to the control only treated with LPS.

4) Effect on Expression of IL-1β Genes

The assay was performed to investigate whether EHMTIQ (11a) affects IL-β production in an activated microglial cell. LPS-stimulated BV-2 cell smaples were treated with various concentrations of EHMTIQ (11a) and the expression of IL-1β genes was estimated by RT-PCR. The results are illustrated in FIG. 4.

As seen from FIG. 4, LPS induced a significant increase in the mRNA level of IL-1β (26±1 times), which was inversely proportional to the concentration of EHMTIQ (11a). A low concentration (2.5 [symbol] ) of EHMTIQ (11a) decreased the mRNA level to 74±0.7%, which was statistically significant, and 100 [symbol] EHMTIQ (11a) led to a significant decrease in the LPS effect (p>0.05, compared with the non-EHMTIQ treated control).

5) Effect on Expression of COX-2 Genes

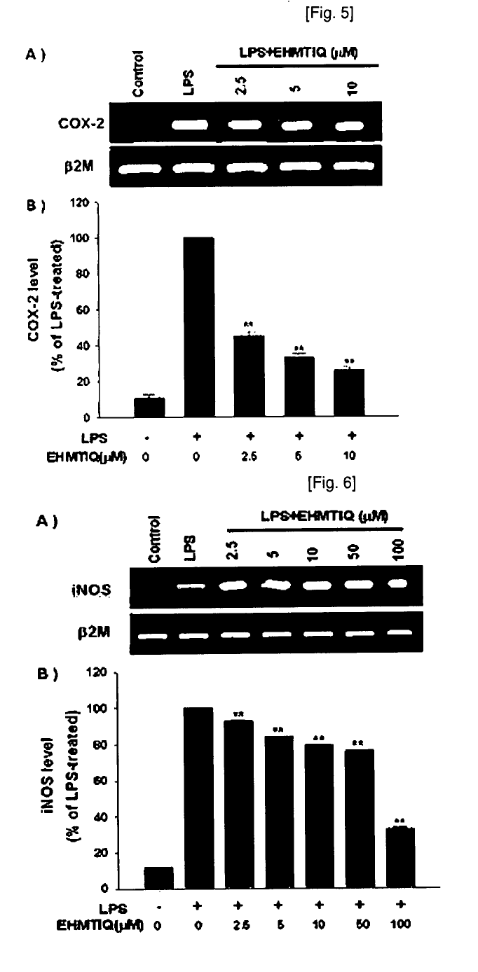

The gene expression of cyclooxigenase-2 (COX-2) stimulating a prostaglandin synthesis causing oxidative stress to neurons increased by the activation of a microglial cell. LPS-stimulated BV-2 cell samples were treated with various concentrations of EHMTIQ (11a), and the expression of COX-2 genes was estimated by RT-PCR. The results are illustrated in FIG. 5.

As seen from FIG. 5, LPS induced a significant increase in the mRNA level of COX-2, which was inversely proportional to the concentration of EHMTIQ (11a). It was confirmed that 2.5 [symbol] , 5 [symbol] and 10 [symbol] EHMTIQ (11a) decreased the COX-2 expression to 62±3%, 75±3% and 83±2%, respectively.

6) Effect on Expression of iNOS Genes

The assay was performed to determine whether EHMTIQ (11a) affects the expression of iNOS genes. LPS-stimulated BV-2 cell samples were treated with various concentrations of EHMTIQ (11a), and the expression of iNOS gene was estimated by RT-PCR. The results are illustrated in FIG. 6.

As seen from FIG. 6, LPS induced a significant increase of 8.5 times in the expression of iNOS genes compared to the control. Such an increase was inhibited by treatment with 5 [symbol] and 100 [symbol] EHMTIQ (11a) to 82±1% and 24±1%, respectively compared to the control only treated with LPS.

7) Effect on Expression of GTPCH Genes

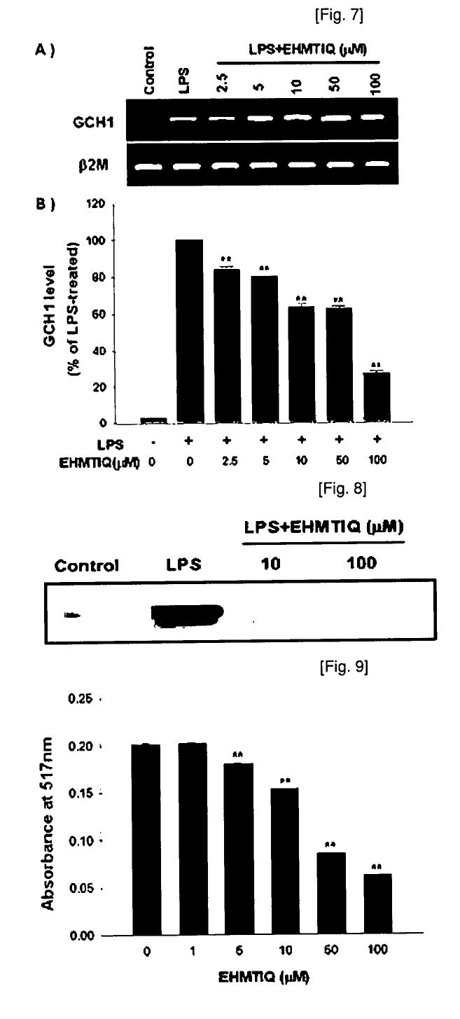

GTP cyclohydrolase I (GTPCH) is a rate-limiting enzyme in the synthesis of tetrahydrobiopterin (BH₄) which is essential for iNOS catalysis. Accordingly, the down regulation of GTPCH may lower NO production. The assay was performed to determine whether EHMTIQ (11a) affects the expression of GTPCH genes induced by LPS. The LPS-stimulated BV-2 cell samples were treated with various concentrations of EHMTIQ (11a) and the expression of GTPCH genes was estimated by RT-PCR. The results are illustrated in FIG. 7.

As seen from FIG. 7, the mRNA level of GRPCH was increased 36.2 times by LPS, but inversely proportional to the concentration of EHMTIQ (11a). A low concentration (2.5 [symbol] ) of EHMTIQ (11a) may decrease the gene expression of GTPCH to 17±1%, and 100 [symbol] EHMTIQ (11a) to 75±1%. The EHMTIQ (11a) itself did not directly relate to the catalysis of GTPCH (not illustrated).

8) Effect on NF-kB Shift to Nucleus

A transcription factor, NF-kB, shifts into a nucleus to regulate expression of several inflammatory genes. Accordingly, the assay was performed to determine whether EHMTIQ (11a) inhibits the NF-kB shift to a nucleus. Samples of cells were treated with LPS only or both LPS and various concentrations of EHMTIQ (11a), and each nuclear fraction was subjected to electrophoresis and Western blot for analyzing the NF-kB p65. The results are illustrated in FIG. 8.

As seen in FIG. 8, while not detected in the control, the NF-kB expression increased in the LPS-treated sample, which, however, was completely inhibited in the presence of 10 [symbol] EHMTIQ (11a).

9) Free Radical Scavenging Activity

Free radicals produced by an activated microglial cell cause oxidative stress and structural transformation in protein, nucleic acid and lipids of a neuron, which lead to cell injury. Accordingly, the assay was performed to determine whether EHMTIQ (11a) has free radical scavenging activity. As seen from FIG. 9, the scavenging activity of DPPH radicals was proportional to the concentration of EHMTIQ (11a).

10) Protective Effect Against Neuron Death Due to Immunological Injury

The assay was performed to determine whether EHMTIQ (11a) protects a dopaminergic cell from injuries due to inflammatory substances released from an activated microglial cell. SK-N-BE(2)C cells were transferred to a culture medium containing substances released from LPS-stimulated BV-2 cells, and the cell death rate was measured by activity of LDH contained in the culture medium and compared with that in the EHMTIQ (11a)-treated BV-2 culture medium.

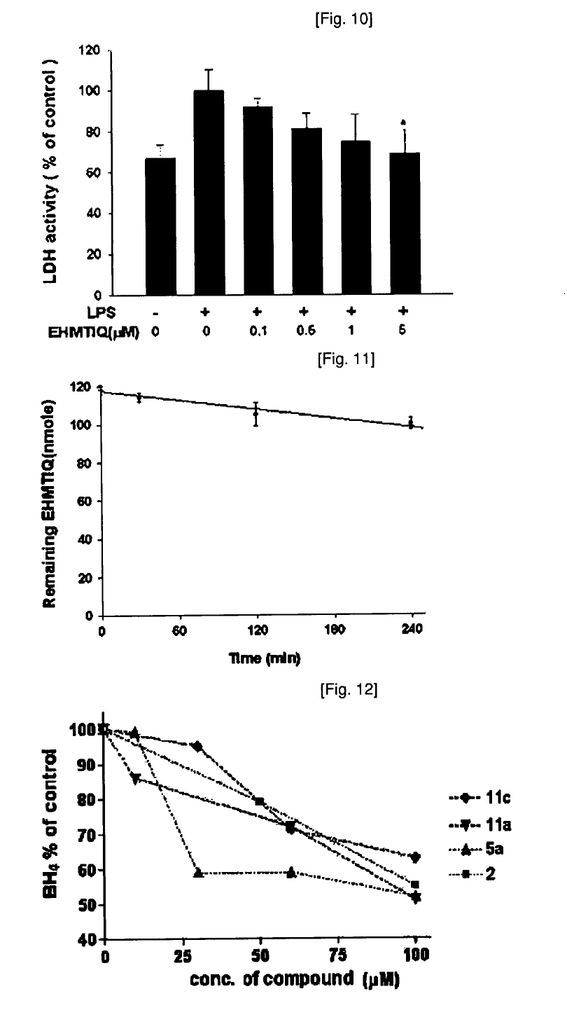

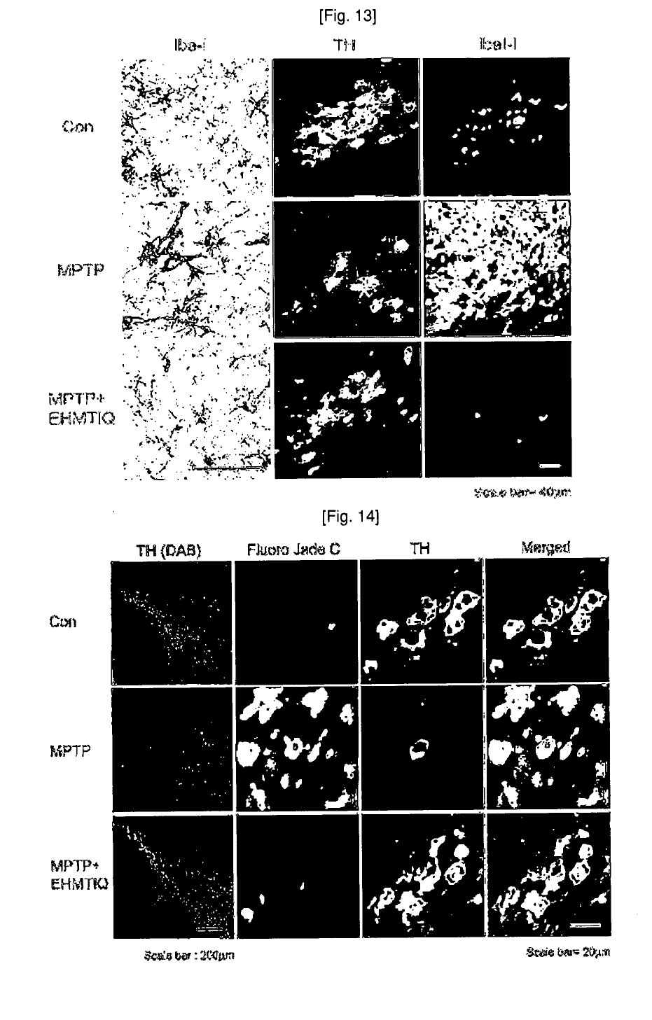

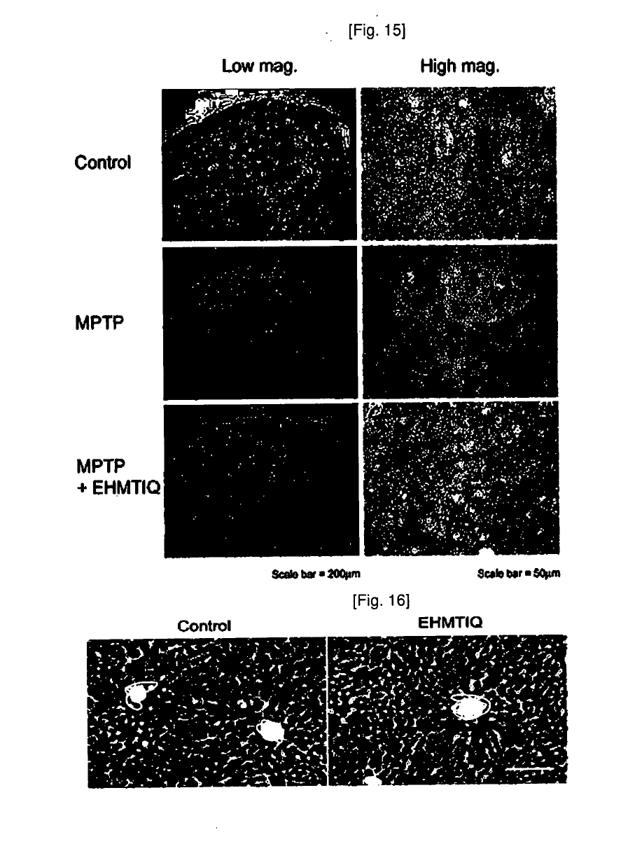

As seen from FIG. 10, 49±10% of the SK-N-BE(2)C cells were injured in the culture medium containing the substances released from the LPS-stimulated BV-2 cells. However, when the SK-N-BE(2)C cells were treated with culture medium obtained from 50 EHMTIQ (11a)+LPS-treated BV-2 cells, the cell injury rate was reduced (p>0.05, compared with non-EHMRIQ treated control).

11) Drug Stability

Since almost all micromolecules are degraded by enzymes in the liver, the stability of a medicine to these enzymes is very important. To evaluate bioavailability of EHMTIQ (11a) in vivo, the degradation rate of EHMTIQ (11a) by liver microsomal enzyme was measured, and neutralized to remaining EHMTIQ after exposure to liver microsomes.

As seen from FIG. 11, almost 95% of EHMTIQ (11a) remained after a 30-minute exposure, which shows that this compound is considerably stable against liver enzyme. After a 2-hour exposure, about 12.52% of EHMTIQ was degraded, which shows that it may be completely degradable in vivo by the liver enzyme given sufficient time. The degeneration rate of EHMTIQ was calculated as 1.115±0.203 nmole (EHMTIQ)/min/mg (liver microsomal protein).

### Exemplary Embodiment 4

### Inhibitory Effect on NO and BH₄ Productions by TIO Derivatives

20 kinds of tetrahydroisoquinoline (TIQ) derivatives were synthesized according to the described methods, and effects of NO and BH₄ productions and cytotoxicity on BV-2 activated microglial cells were assayed as follows.

### INDUSTRIAL APPLICABILITY

Consequently, compounds of the present invention are effective as medicines in treating inflammatory and neurodegenerative diseases.

While the invention has been shown and described with reference to certain exemplary embodiments thereof, it will be understood by those skilled in the art that various changes in form and details may be made therein without departing from the spirit and scope of the invention as defined by the appended claims.

1. A 7-hydroxy-6-methoxy-1,2,3,4-tetrahydroisoquinoline derivative of Formula 1, 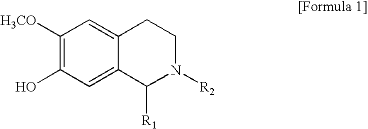
wherein R₁ is selected from the group consisting of H, CH₃, CH₂CH₃, CH₂CH₂CH₃, CH(CH₃)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH₂CH₃, CH₂CH₂CH₂CH₃, CH₂Ph, CH₂CH₂Ph, COCH₂CH₃, COCH₂CH₂CH₃, COCH(CH₃)₂, COCH₂CH(CH₃)₂, cyclohexylmethyl and cyclohexanecarbonyl.

2-14. (canceled)

15. The derivative according to claim 1, wherein R₁ is H, and R₂ is COCH₂CH₃.

16. The derivative according to claim 1, wherein R₁ is H, and R₂ is COCH₂CH₂CH₃.

17. The derivative according to claim 1, wherein R₁ is H, and R₂ is COCH(CH₃)₂.

18. The derivative according to claim 1, wherein R₁ is H, and R₂ is COCH₂CH(CH₃)₂.

19. The derivative according to claim 1, wherein R₁ is H, and R₂ is cyclohexanecarbonyl.

20. The derivative according to claim 1, wherein R₁ is H, and R₂ is CH₂CH₃.

21. The derivative according to claim 1, wherein R₁ is H, and R₂ is CH₂CH₂CH₃.

22. The derivative according to claim 1, wherein R₁ is H, and R₂ is CH₂CH₂CH₂CH₃.

23. The derivative according to claim 1, wherein R₁ is H, and R₂ is cyclohexylmethyl.

24. The derivative according to claim 1, wherein R₁ is H, and R₂ is CH₂Ph.

25. The derivative according to claim 1, wherein R₁ is H, and R₂ is CH₂CH₂Ph.

26. (canceled)

27. A pharmaceutical composition for preventing and treating degenerative diseases comprising 7-hydroxy-6-methoxy-1,2,3,4-tetrahydroisoquinoline derivative of Formula 1, 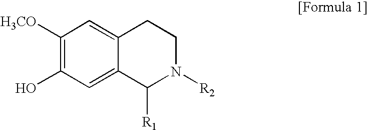
wherein R₁ is selected from the group consisting of H, CH₃, CH₂CH₃, CH₂CH₂CH₃, CH(CH₃)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH_{Z}CH₃, CH₂CH₂CH₂CH₃, CH₂Ph, CH₂CH₂Ph, COCH₃(Ac), COCH₂CH₃, COCH₂CH₂CH₃, COCH(CH₃)₂, COCH₂CH(CH₃)₂, cyclohexylmethyl and cyclohexanecarbonyl.

28. The pharmaceutical composition according to claim 27, wherein the degenerative diseases include neurodegenerative diseases and arthritis.

29. A pharmaceutical composition for preventing and treating inflammatory diseases comprising 7-hydroxy-6-methoxy-1,2,3,4-tetrahydroisoquinoline derivative of Formula 1, 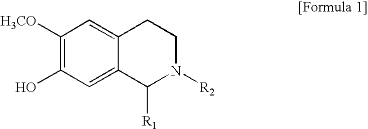
wherein R₁ is selected from the group consisting of H, CH₃, CH₂CH₃, CH₂CH₂CH₃, CH(CH₃)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH₂CH₃, CH₂CH₂CH₂CH₃, CH₂Ph, CH₂CH₂Ph, COCH₃(Ac), COCH₂CH₃, COCH₂CH₂CH₃, COCH(CH₃)₂, COCH₂CH(CH₃)₂, cyclohexylmethyl and cyclohexanecarbonyl.

30. A pharmaceutical composition having effects of protecting neurons comprising 7-hydroxy-6-methoxy-1,2,3,4-tetrahydroisoquinoline derivative of Formula 1, 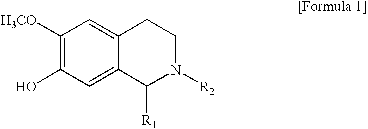
wherein R₁ is selected from the group consisting of H, CH₃, CH₂CH₃, CH₂CH₂CH₃, CH(CH₃)₂, CH₂CH(CH₃)₂, Ph, CH₂Ph, cyclobutyl, cyclopropyl and cyclohexyl, and R₂ is selected from the group consisting of CH₃, CH₂CH₃, CH₂CH₂CH₃, CH₂CH₂CH₂CH₃, CH₂Ph, CH₂CH₂Ph, COCH₃(Ac), COCH₂CH₃, COCH₂CH₂CH₃, COCH(CH₃)₂, COCH₂CH(CH₃)₂, cyclohexylmethyl and cyclohexanecarbonyl.


SEQUENCE LISTING (10 sequences)

SEQ ID NO: 1 - DNA, 20 bp, Artificial Sequence
  forward primer for iNOS
atgtccgaagcaaacatcac

SEQ ID NO: 2 - DNA, 20 bp, Artificial Sequence
  backward primer for iNOS
taatgtccaggaagtaggtg

SEQ ID NO: 3 - DNA, 25 bp, Artificial Sequence
  forward primer for TNF-a
cagaccctcacactcagatcatctt

SEQ ID NO: 4 - DNA, 26 bp, Artificial Sequence
  backward primer for TNF-a
cagagcaatgactccaaagtagacct

SEQ ID NO: 5 - DNA, 25 bp, Artificial Sequence
  forward primer for IL-1b
atggcaactgttcctgaactcaact

SEQ ID NO: 6 - DNA, 26 bp, Artificial Sequence
  backward primer for IL-1b
caggacaggtatagattctttccttt

SEQ ID NO: 7 - DNA, 20 bp, Artificial Sequence
  forward primer for COX-2
cagcaaatccttgctgttcc

SEQ ID NO: 8 - DNA, 21 bp, Artificial Sequence
  backward primer for COX-2
tgggcaaagaatgcaaacatc

SEQ ID NO: 9 - DNA, 21 bp, Artificial Sequence
  forward primer for GTPCH
ggataccaggagaccatctca

SEQ ID NO: 10 - DNA, 21 bp, Artificial Sequence
  backward primer for GTPCH
tagcatggtgctagtgacagt